(19) United States
(12) Patent Application Publication
(10) Pub. No.: US 2009/0312311 A1
(43) Pub. Date: Dec. 17, 2009

(54) COMBINATION OF ORGANIC COMPOUNDS

(76) Inventors: Gary Michael Ksander, Amherst, NH (US); Suraj Shivappa Shetty, Andover, MA (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/225,958

(22) PCT Filed: Apr. 4, 2007
(86) PCT No.: PCT/US2007/065912

§ 371 Date: Oct. 3, 2008

Related U.S. Application Data
(60) Provisional application No. 60/789,831, filed on Apr. 6, 2006.

(51) Int. Cl. A61K 31/5415; A61P 9/00; A61P 9/04

(52) U.S. Cl. 514/223.5

(57) ABSTRACT

The present invention relates to a combination of organic compounds, a pharmaceutical composition and a kit of parts comprising said combination of organic compounds and to a method of treatment or prevention of certain conditions or diseases

The present invention relates to a combination of organic compounds that are useful for the treatment and/or prevention of cardiovascular disorders including pathologic cardiac hypertrophy and heart failure

Reversible acetylation of histones is a major regulator of gene expression that acts by altering accessibility of transcription factors to DNA. In normal cells, histone deacetylase (HDAC) and histone acetyltrasferase together control the level of acetylation of histones to maintain a balance. Inhibition of HDAC results in the accumulation of hyperacetylated histones, which results in a variety of cellular responses.

Inhibitors of HDAC have been studied for their therapeutic effects on cancer cells. For example, butyric acid and its derivatives, including sodium phenylbutyrate, have been reported to induce apoptosis in vitro in human colon carcinoma, leukemia and retinoblastoma cell lines. However, butyric acid and its derivatives are not useful pharmacological agents because they tend to be metabolized rapidly and have a very short half-life in vivo. Other inhibitors of HDAC that have been widely studied for their anti-cancer activities are trichostatin A and trapoxin. Trichostatin A is an antifungal and antibiotic and is a reversible inhibitor of mammalian HDAC. Trapoxin is a cyclic tetrapeptide, which is an irreversible inhibitor of mammalian HDAC. Although trichostatin and trapoxin have been studied for their anti-cancer activities, the in vivo instability of the compounds makes them less suitable as anti-cancer drugs.

Inhibitors of HDAC have also been studied for their therapeutic effects on pathological cardiac hypertrophy and heart failure. Transgenic mice that overexpress Hop, a homeodomain protein expressed by cardiac myocytes, develop severe cardiac hypertrophy, cardiac fibrosis, and premature death. Treatment of these animals with trichostatin A, an HDAC inhibitor, prevents cardiac hypertrophy (Kook et al. 2003). In addition, trichostatin A also attenuates hypertrophy induced by infusion of isoproterenol.

On the other hand, Angiotensin (Ang) II is a key player in left ventricular (LV) remodeling and cardiac fibrosis. Its effects are thought to be mediated at least in part by mitogen-activated protein kinases (MAPK), transforming growth factor (TGF) beta1, and the Smad pathway. In recent times great efforts have been made to identify substances that antagonize the AT₁-receptor. Such active ingredients are often called as angiotensin II antagonists or angiotensin II blockers (ARBs). As a result of the inhibition of the AT₁-receptor activity such antagonists may also be employed, e.g., as antihypertensives or for the treatment of congestive heart failure, among other indications. Angiotensin II blockers are therefore understood to be those active agents which bind to the AT₁-receptor subtype but do not result in activation of the receptor. Further evaluations have revealed that angiotensin II blockers may also be employed for a much broader range of therapeutic indications.

The treatment of heart failure (HF) may be divided into four components: (1) removal of the precipitating cause, (2) correction of the underlying cause, (3) prevention of deterioration of cardiac function, and (4) control of the congestive HF state.

Conventionally, HF has been treated with a wide variety of drugs, including alpha-adrenergic agonists, beta-adrenergic antagonists, calcium channel antagonists, cardiac glycosides, diuretics, nitrates, phosphodiesterase inhibitors, prazosin, and a variety of vasodilators.

All of these drugs, however, have undesirable side-effects. For example, use of alpha-adrenergic agonists results in edema of the peripheral tissues. The prolonged use of β-adrenergic agents leads to the progressive development of desensitization to the drug. Cardiac glycosides produce toxic side-effects in the CNS, and also in the gastrointestinal and respiratory systems. They additionally produce pro-arrhythmic effects. Treatment with diuretics may result in a variety of adverse-effects, such as hyponatremia, hypokalemia, and hyperchloremic metabolic alkalosis.

Prolonged use of calcium channel antagonists, such as verapamil, diltiazem and nifedipine. render them ineffective. Moreover, calcium channel antagonists have been shown to increase the mortality rates in patients thus treated, because such compounds act to increase oxygen consumption, which further stresses the compromised heart.

Thus, there is a continued demand for new, non toxic, compounds for treating HF, improving left ventricle function, without increasing the myocardial oxygen requirement. It is also preferred that the drugs do not act directly to stimulate cardiac contractility, or produce side-effects such as changes in blood pressure and/or heart rate, since they are associated with increased mortality in patients with HF.

Although therapeutic agents currently under study appear to be promising, a number of factors may render them at present less suitable as a treatment option for cardiovascular disorders, in particular heart failure. The nature of cardiovascular disorders is multifactorial, and under certain circumstances, therapeutic agents with different mechanism of action have been combined. However, just considering any combination of drugs having different mode of action does not necessarily lead to drug combinations with advantageous effects. Accordingly, there is an urgent need to identify more efficacious therapies, in particular combination therapies, which have less deleterious side effects for the treatment of cardiovascular disorders.

Thus, there exists a strong need for further development of combinations and pharmaceutical compositions that are suitable for treating and/or preventing pathological cardiac hypertrophy and ameliorating or reversing the biochemical processes that lead to heart failure and death.

Thus, the invention further relates to a combination comprising

(i) an angiotensin receptor blocker (ARB) or a pharmaceutically acceptable salt thereof, and
(ii) a histone deacetylase (HDAC) inhibitor or a pharmaceutically acceptable salt thereof.

Listed below are some of the definitions of various additional terms used herein to describe certain aspects of the present invention. However, the definitions used herein are those generally known in the art, e.g., hypertension, heart failure and atherosclerosis, and apply to the terms as they are used throughout the specification unless they are otherwise limited in specific instances.

The term “prevention” refers to prophylactic administration to healthy patients to prevent the development of the conditions mentioned herein. Moreover, the term “prevention” means prophylactic administration to patients being in a pre-stage of the conditions to be treated.

The term “delay the onset of”, as used herein, refers to administration to patients being in a pre-stage of the condition to be treated in which patients with a pre-form of the corresponding condition is diagnosed.

The term “treatment” is understood the management and care of a patient for the purpose of combating the disease, condition or disorder.

The term “therapeutically effective amount” refers to an amount of a drug or a therapeutic agent that will elicit the desired biological or medical response of a tissue, system or an animal (including man) that is being sought by a researcher or clinician.

The term “synergistic”, as used herein, means that the effect achieved with the methods, combinations and pharmaceutical compositions of the present invention is greater than the sum of the effects that result from individual methods and compositions comprising the active ingredients of this invention separately.

The term “warm-blooded animal or patient” are used interchangeably herein and include, but are not limited to, humans, dogs, cats, horses, pigs, cows, monkeys, rabbits, mice and laboratory animals. The preferred mammals are humans.

The term “pharmaceutically acceptable salt” refers to a non-toxic salt commonly used in the pharmaceutical industry which may be prepared according to methods well-known in the art.

The term “type 2 diabetes” including type 2 diabetes associated with hypertension refers to a disease in which the pancreas does not secrete sufficient insulin due to an impairment of pancreatic beta-cell function and/or in which there is to insensitivity to produced insulin (insulin resistance). Typically, the fasting plasma glucose is less than 126 mg/dL, while pre-diabetes is, e.g., a condition which is characterized by one of following conditions: impaired fasting glucose (110-125 mg/dL) and impaired glucose tolerance (fasting glucose levels less than 126 mg/dL and post-prandial glucose level between 140 mg/dL and 199 mg/dL). Type 2 diabetes mellitus can be associated with or without hypertension. Diabetes mellitus occurs frequently, e.g., in African American, Latino/Hispanic American, Native American, Native American, Asian American and Pacific Islanders. Markers of insulin resistance include HbA1C, HOMA IR, measuring collagen fragments, TGF-□ in urine, PAI-1 and prorenin.

The term “hypertension” refers to a condition where the pressure of blood within the blood vessels is higher than normal as it circulates through the body. When the systolic pressure exceeds 150 mmHg or the diastolic pressure exceeds 90 mmHg for a sustained period of time, damage is done to the body. For example, excessive systolic pressure can rupture blood vessels anywhere, and when it occurs within the brain, a stroke results. Hypertension may also cause thickening and narrowing of the blood vessels which ultimately could lead to atherosclerosis.

The term “severe hypertension” refers to hypertension characterized by a systolic blood pressure of ≧180 mmHg and a diastolic blood pressure of ≧110 mmHg.

The term “pulmonary hypertension” (PH) refers to a blood vessel disorder of the lung in which the pressure in the pulmonary artery rises above normal level of ≦25/10 (especially primary and secondary PH), e.g., because the small vessels that supply blood to the lungs constrict or tighten up. According to the WHO, PH may be divided into five categories: pulmonary arterial hypertension (PAH), a PH occurring in the absence of a known cause is referred to as primary pulmonary hypertension, while secondary PH is caused by a condition selected, e.g., from emphysema; bronchitis; collagen vascular diseases, such as scleroderma, Crest syndrome or systemic lupus erythematosus (SLE); PH associated with disorders of the respiratory system; PH due to chronic thrombotic or embolic disease; PH due to disorders directly affecting the pulmonary blood vessels; and pulmonary venous hypertension (PVH).

The term “malignant hypertension” is usually defined as very high blood pressure with swelling of the optic nerve behind the eye, called papilledema (grade IV Keith-Wagner hypertensive retinopathy). This also includes malignant HTN of childhood.

The term “isolated systolic hypertension” refers to hypertension characterized by a systolic blood pressure of ≧140 mmHg and a diastolic blood pressure of <90 mmHg.

The term “familial dyslipidemic hypertension” is characterized by mixed dyslipidemic disorders. Biomarkers include oxidized LDL, HDL, glutathione and homocysteine LPa.

The term “renovascular hypertension” (renal artery stenosis) refers to a condition where the narrowing of the renal artery is significant which leads to an increase of the blood pressure resulting from signals sent out by the kidneys. Biomarkers include renin, PRA and prorenin.

The term “endothelial dysfunction” with or without hypertension refers to a condition in which normal dilation of blood vessels is impaired due to lack of endothelium-derived vasodilators. Biomarkers include CRP, IL6, ET1, BIG-ET1, VCAM and ICAM. Survival post-MI biomarkers include BNP and procollagen factors.

The term “diastolic dysfunction” refers to abnormal mechanical properties of the heart muscle (myocardium) and includes abnormal left ventricle (LV) diastolic distensibility, impaired filling, and slow or delayed relaxation regardless of whether the ejection fraction is normal or depressed and whether the patient is asymptomatic or symptomatic. Asymptomatic diastolic dysfunction is used to refer to an asymptomatic patient with a normal ejection fraction and an abnormal echo-Doppler pattern of LV filling which is often seen, for example, in patients with hypertensive heart disease. Thus, an asymptomatic patient with hypertensive left ventricular hypertrophy and an echocardiogram showing a normal ejection fraction and abnormal left ventricular filling can be said to have diastolic dysfunction. If such a patient were to exhibit symptoms of effort intolerance and dyspnea, especially if there were evidence of venous congestion and pulmonary edema, it would be more appropriate to use the term diastolic heart failure. This terminology parallels that used in asymptomatic and symptomatic patients with LV systolic dysfunction, and it facilitates the use of a pathophysiologic, diagnostic, and therapeutic framework that includes all patients with LV dysfunction whether or not they have symptoms (William H. Gaasch and Michael R. Zile, Annu. Rev. Med. 55: 373-94, 2004; Gerard P. Aurigemma, William H. Gaasch, N. Engl. J. Med. 351:1097-105, 2004).

The term “cardiac fibrosis” is defined as abnormally high accumulation of collagen and other extracellular matrix proteins due to the enhanced production or decreased degradation of these proteins. Biomarkers include BNP, procollagen factors, LVH, AGE RAGE and CAGE.

The term “peripheral vascular disease” (PVD) refers to the damage or dysfunction of peripheral blood vessels. There are two types of peripheral vascular diseases: peripheral arterial disease (PAD) which refers to diseased peripheral arteries and peripheral venous disorders, which can be measured by an ankle brachial index. PAD is a condition that progressively hardens and narrows arteries due to a gradual buildup of plaque and refers to conditions that effect the blood vessels, such as arteries, veins and capillaries, of the body outside the heart. This is also known as peripheral venous disorder.

The term “atherosclerosis” comes from the Greek words athero (meaning gruel or paste) and sclerosis (hardness). It's the name of the process in which deposits of fatty substances, cholesterol, cellular waste products, calcium and other substances build up in the inner lining of an artery. This buildup is called plaque. It usually affects large and medium-sized arteries. Some hardening of arteries often occurs when people grow older. Plaques can grow large enough to significantly reduce the blood's flow through an artery. But most of the damage occurs when they become fragile and rupture. Plaques that rupture cause blood clots to form that can block blood flow or break off and travel to another part of the body. If either happens and blocks a blood vessel that feeds the heart, it causes a heart attack. If it blocks a blood vessel that feeds the brain, it causes a stroke. And if blood supply to the arms or legs is reduced, it can cause difficulty walking and eventually gangrene.

The term “coronary arterial disease” (CAD) also refers to a condition that progressively hardens and narrows arteries due to a gradual buildup of plaque and refers to conditions that effect the blood vessels such as arteries within the heart. CAD is peculiar form of atherosclerosis that occurs in the three small arteries supplying the heart muscle with oxygen-rich blood. Biomarkers include CPK and Troponin.

The term “cerebrovascular diseases” comprise stroke conditions, such as embolic and thrombotic stroke; large vessel thrombosis and small vessel disease; and hemorrhagic stroke.

The term “embolic stroke” refers to a condition characterized by the formation of blood clots, e.g., in the heart, when clots travel down through the bloodstream in the brain. This may lead to a blockade of small blood vessels and causing a stroke.

The term “thrombotic stroke” refers to a condition where the blood flow is impaired because of a blockade to one or more of the arteries supplying blood to the brain. This process normally leads to thrombosis causing thrombotic strokes. Biomarkers include PAI 1, TPA and platelet function.

The term “metabolic syndrome” (Syndrome X) refers to an overall condition characterized by three or more of the following criteria:
1. abdominal obesity: waist circumference>102 cm in men, and >88 cm in women;
2. hypertriglyceridemia: >150 mg/dL (1.695 mmol/L);
3. low HDL cholesterol: <40 mg/dL (1.036 mmol/L) in men, and <50 mg/dL (1.295 mmol/L) in women;
4. high blood pressure: >130/85 mmHg; and
5. high-fasting glucose: >110 mg/dL (>6.1 mmol/L).

Metabolic syndrome may also be characterized by three or more of the following criteria: triglycerides>150 mg/dL, systolic blood pressure (BP)≧130 mmHg or diastolic BP≧85 mmHg, or on anti-hypertensive treatment, high-density lipoprotein cholesterol<40 mg/dL, fasting blood sugar (FBS)>110 mg/dL, and a body mass index (BMI)>28.8 k/m².

Metabolic syndrome may also be characterized by diabetes, impaired glucose tolerance, impaired fasting glucose, or insulin resistance plus two or more of the following abnormalities:
1. high blood pressure: ≧160/90 mmHg;
2. hyperlipidemia: triglyceride concentration≧150 mg/dL (1.695 mmol/L) and/or HDL cholesterol<35 mg/dL (0.9 mmol/L) in men, and <39 mg/dL (1.0 mmol/L) in women;
3. central obesity: waist-to-hip ratio of >0.90 in men, and >0.85 in women and/or BMI>30 kg/m²; and
4. microalbuminuria: urinary albumin excretion rate≧20 μg/min or an albumin-to-creatinine ratio≧20 mg/g. Biomarkers include proteinuria, TGF-β, TNF-α and adiponectin.

Biomarkers include LDL, HDL and all the endothelial dysfunction markers.

The term “atrial fibrillation” (AF) refers to a type of irregular or racing heartbeat that may cause blood to collect in the heart and potentially form a clot which may travel to the brain and can cause a stroke.

The term “renal failure”, e.g., chronic renal failure; is characterized, e.g., by proteinuria and/or slight elevation of plasma creatinine concentration (106-177 mmol/L corresponding to 1.2-2.0 mg/dL).

The term “glomerulonephritis” refers to a condition which may be associated with the nephrotic syndrome, a high blood pressure and a decreased renal function, focal, segmental glomerulonephritis, minimal change nephropathy, Lupus nephritis, post-streptococcal GN and IgA nephropathy.

The term “nephrotic syndrome” refers to a compilation of conditions including massive proteinuria, edema and central nervous system (CNS) irregularities. Biomarkers include urinary protein excretion.

The term “plaque stabilization” means rendering a plaque less dangerous by preventing, fibrous cap thinning/rupture, smooth muscle cell loss and inflammatory cell accumulation.

The term “renal fibrosis” refers to an abnormal accumulation of collagen and other extracellular matrix proteins, leading to loss of renal function. Biomarkers include collagen fragments and TGF-β in urine.

The term “end-stage renal disease” (ESRD) refers to loss of renal function to the extent that dialysis or renal replacement is needed. Biomarkers include glomerular filtration rate and creatinine clearance.

The term “polycystic kidney disease” (PKD) refers to a genetic disorder characterized by the growth of numerous cysts in the kidney. PKD cysts can slowly reduce much of the mass of kidneys reducing kidney function and leading to kidney failure. PKD may be classified as two major inherited forms of PKD which are autosomal dominant PKD and autosomal recessive PKD, while the non-inherited PKD may be called acquired cystic kidney disease. Biomarkers include reduction of renal cysts by non-invasive imaging.

Congestive heart failure (CHF), or heart failure (HF), is a term used to describe any condition in which the heart is unable to adequately pump blood throughout the body and/or unable to prevent blood from “backing up” into the lungs. These conditions cause symptoms such as shortness of breath (dyspnea), fatigue, weakness, and swelling (edema) of the legs and sometimes the abdomen.

Congestive heart failure, regardless of its etiology, is characterized by a weakness of the myocardial tissue of the left and/or right ventricles of the heart and the resulting difficulty in pumping and circulating blood to the systemic and/or pulmonary systems. Myocardial tissue weakness is typically associated with circulatory and neurohumoral changes which result in a failure to deliver sufficient blood and oxygen to peripheral tissues and organs. Some of the resulting changes include higher pulmonary and systemic pressure, lower cardiac output, higher vascular resistance and peripheral and pulmonary edema. Congestive heart failure may be further expressed as shortness of breath either on exertion, at rest or paroxysmal nocturnal dyspnea. If left untreated, congestive heart failure can lead to death.

Heart failure may be described as systolic or diastolic, high-output or low-output, acute or chronic, right-sided or left-sided, and forward or backward. These descriptors are often useful in a clinical setting, particularly early in the patient's course, but late in the course of chronic HF the differences between them often become blurred.

Systolic Versus Diastolic Failure: The distinction between these two forms of HF, relates to whether the principal abnormality is the inability of the ventricle to contract normally and expel sufficient blood (systolic failure) or to relax and/or fill normally (diastolic failure).

High-Output versus Low-Output Heart Failure: It is useful to classify patients with HF into those with a low cardiac output, i.e., low-output HF, and those with an elevated cardiac output, i.e., high-output HF.

Acute versus Chronic Heart Failure: The prototype of acute HF is the sudden development of a large myocardial infarction or rupture of a cardiac valve in a patient who previously was entirely well. Chronic HF is typically observed in patients with dilated cardiomyopathy or multivalvular heart disease that develops or progresses slowly. Acute HF is usually predominantly systolic, and the sudden reduction in cardiac output often results in systemic hypotension without peripheral edema. In contrast, in chronic HF, arterial pressure is ordinarily well maintained until very late in the course, but there is often accumulation of edema.

Right-Sided versus Left-Sided Heart Failure: Many of the clinical manifestations of HF result from the accumulation of excess fluid behind either one or both ventricles. This fluid usually localizes upstream to (behind) the ventricle that is initially affected.

Backward versus Forward Heart Failure: For many years a controversy has revolved around the question of the mechanism of the clinical manifestations resulting from HF. A rigid distinction between backward and forward HF (like a rigid distinction between right and left HF) is artificial, since both mechanisms appear to operate to varying extents in most patients with HF.

The term “combination” of an angiotensin receptor blocker (ARB) or a pharmaceutically acceptable salt thereof and a histone deacetylase (HDAC) inhibitor or a pharmaceutically acceptable salt thereof means that the components can be administered together as a pharmaceutical composition or as part of the same, unitary dosage form. A combination also includes administering an angiotensin receptor blocker (ARB) or a pharmaceutically acceptable salt thereof and a histone deacetylase (HDAC) inhibitor or a pharmaceutically acceptable salt thereof each separately but as part of the same therapeutic regimen. The components, if administered separately, need not necessarily be administered at essentially the same time, although they can if so desired. Thus, a combination also refers, for example, administering an angiotensin receptor blocker (ARB) or a pharmaceutically acceptable salt thereof and a histone deacetylase (HDAC) inhibitor or a pharmaceutically acceptable salt thereof as separate dosages or dosage forms, but at the same time. A combination also includes separate administration at different times and in any order. Suitable angiotensin II receptor blockers which may be employed in the combination of the present invention include AT₁-receptor antagonists having differing structural features, preferred are those with the non-peptidic structures. For example, mention may be made of the compounds that are selected from the group consisting of valsartan (EP 443983), losartan (EP 253310), candesartan (EP 459136), eprosartan (EP 403159), irbesartan (EP 454511), olmesartan (EP 503785), tasosartan (EP 539086), telmisartan (EP 522314), saprisartan, the compound with the designation E-4177 of the formula

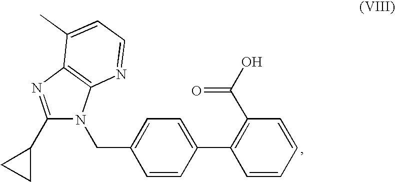

the compound with the designation SC-52458 of the following formula

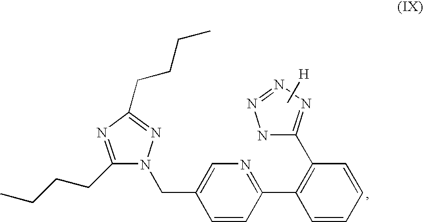

and the compound with the designation the compound ZD-8731 of the formula

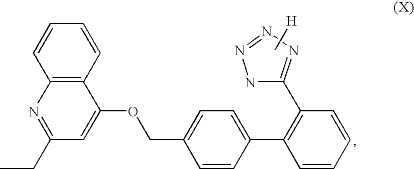

or, in each case, a pharmaceutically acceptable salt thereof.

Preferred AT₁-receptor antagonists are those agents that have reached the market, most preferred is valsartan, or a pharmaceutically acceptable salt thereof.

Suitable histone deacetylase (HDAC) inhibitors which may be employed in the combination of the present invention include those HDAC inhibitors that have been or are developed in oncology. For example, mention may be made of the compounds that are selected from the group consisting of AN-9 [Pivaloyloxymethyl Butyrate, Pivanex®] having the structure

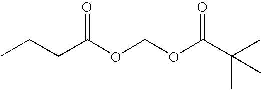

as disclosed in EP-A-00302349; FK-228 having the structure

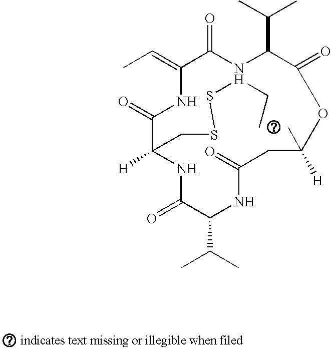

as disclosed in EP-A-00352646; suberoylanilide hydroxamic acid (“SAHA”) having the structure

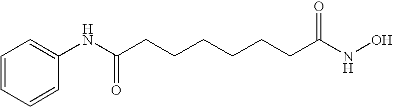

as disclosed in WO2000118171; MGCD-0103 having the structure

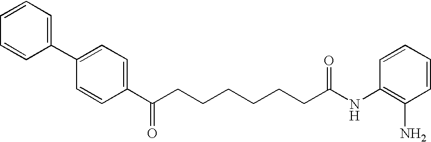

[symbol]

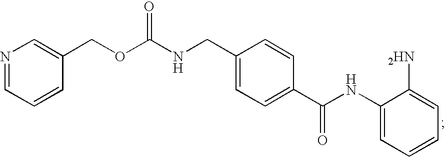

[symbol]

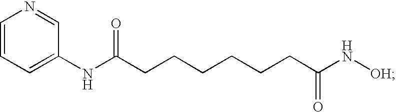

tacedinaline having the structure

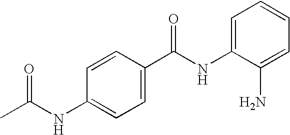

as disclosed in DE03613571, WO2000018393 or WO2000134131; and PXD-101 having the structure

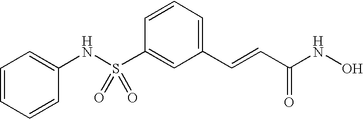

as disclosed in WO2000230879 and US06888027;
or a compound of formula (I) as disclosed in WO200222577

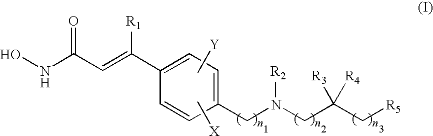

wherein
R₁ is H, halo, or a straight chain C₁-C₆ alkyl (especially methyl, ethyl or n-propyl, which methyl, ethyl and n-propyl substituents are unsubstituted or substituted by one or more substituents described below for alkyl substituents);
R₂ is selected from H, C₁-C₁₀ alkyl, (preferably C₁-C₆ alkyl, e.g. methyl, ethyl or —CH₂CH₂—OH), C₄-C₉ cycloalkyl, C₄-C₉ heterocycloalkyl, C₄-C₉ heterocycloalkylalkyl, cycloalkylalkyl (e.g., cyclopropylmethyl), aryl, heteroaryl, arylalkyl (e.g. benzyl), heteroarylalkyl (e.g. pyridylmethyl), —(CH₂)ₙC(O)R₆, —(CH₂)ₙOC(O)R₆, amino acyl, HON—C(O)—CH═C(R₁)-aryl-alkyl- and —(CH₂)ₙR₇;
R₃ and R₄ are the same or different and independently H, C₁-C₆ alkyl, acyl or acylamino, or R₃ and R₄ together with the carbon to which they are bound represent C═O, C═S, or C═NR₈, or R₂ together with the nitrogen to which it is bound and R₃ together with the carbon to which it is bound can form a C₄-C₉ heterocycloalkyl, a heteroaryl, a polyheteroaryl, a non-aromatic polyheterocycle, or a mixed aryl and non-aryl polyheterocycle ring;
R₅ is selected from H, C₁-C₆ alkyl, C₄-C₉ cycloalkyl, C₄-C₉ heterocycloalkyl, acyl, aryl, heteroaryl, arylalkyl (e.g. benzyl), heteroarylalkyl (e.g. pyridylmethyl), aromatic polycycles, non-aromatic polycycles, mixed aryl and non-aryl polycycles, polyheteroaryl, non-aromatic polyheterocycles, and mixed aryl and non-aryl polyheterocycles;
n, n₁, n₂ and n₃ are the same or different and independently selected from 0-6, when n₁ is 1-6, each carbon atom can be optionally and independently substituted with R₃ and/or R₄;
X and Y are the same or different and independently selected from H, halo, C₁-C₄ alkyl, such as CH₃ and CF₃, NO₂, C(O)R₁, OR₉, SR₉, CN, and NR₁₀R₁₁;
R₆ is selected from H, C₁-C₆ alkyl, C₄-C₉ cycloalkyl, C₄-C₉ heterocycloalkyl, cycloalkylalkyl (e.g., cyclopropylmethyl), aryl, heteroaryl, arylalkyl (e.g., benzyl, 2-phenylethenyl), heteroarylalkyl (e.g., pyridylmethyl), OR₁₂, and NR₁₃R₁₄;
R₇ is selected from OR₁₅, SR₁₅, S(O)R₁₆, SO₂R₁₇, NR₁₃R₁₄, and NR₁₂SO₂R₆;
R₈ is selected from H, OR₁₅, NR₁₃R₁₄, C₁-C₆ alkyl, C₄-C₉ cycloalkyl, C₄-C₉ heterocycloalkyl, aryl, heteroaryl, arylalkyl (e.g., benzyl), and heteroarylalkyl (e.g., pyridylmethyl);
R₉ is selected from C₁-C₄ alkyl, for example, CH₃ and CF₃, C(O)-alkyl, for example C(O)CH₃, and C(O)CF₃;
R₁₀ and R₁₁ are the same or different and independently selected from H, C₁-C₄ alkyl, and —C(O)-alkyl;
R₁₂ is selected from H, C₁-C₆ alkyl, C₄-C₉ cycloalkyl, C₄-C₉ heterocycloalkyl, C₄-C₉ heterocycloalkylalkyl, aryl, mixed aryl and non-aryl polycycle, heteroaryl, arylalkyl (e.g., benzyl), and heteroarylalkyl (e.g., pyridylmethyl);
R₁₃ and R₁₄ are the same or different and independently selected from H, C₁-C₆ alkyl, C₄-C₉ cycloalkyl, C₄-C₉ heterocycloalkyl, aryl, heteroaryl, arylalkyl (e.g., benzyl), heteroarylalkyl (e.g., pyridylmethyl), amino acyl, or R₁₃ and R₁₄ together with the nitrogen to which they are bound are C₄-C₉ heterocycloalkyl, heteroaryl, polyheteroaryl, non-aromatic polyheterocycle or mixed aryl and non-aryl polyheterocycle;
R₁₅ is selected from H, C₁-C₆ alkyl, C₄-C₉ cycloalkyl, C₄-C₉ heterocycloalkyl, aryl, heteroaryl, arylalkyl, heteroarylalkyl and (CH₂)ₘZR₁₂;
R₁₆ is selected from C₁-C₆ alkyl, C₄-C₉ cycloalkyl, C₄-C₉ heterocycloalkyl, aryl, heteroaryl, polyheteroaryl, arylalkyl, heteroarylalkyl and (CH₂)ₘZR₁₂;
R₁₇ is selected from C₁-C₆ alkyl, C₄-C₉ cycloalkyl, C₄-C₉ heterocycloalkyl, aryl, aromatic polycycles, heteroaryl, arylalkyl, heteroarylalkyl, polyheteroaryl and NR₁₃R₁₄;
m is an integer selected from 0 to 6; and
Z is selected from O, NR₁₃, S and S(O),
   or a pharmaceutically acceptable salt thereof.

As appropriate, unsubstituted means that there is no substituent or that the only substituents are hydrogen.

Halo substituents are selected from fluoro, chloro, bromo and iodo, preferably fluoro or chloro.

Alkyl substituents include straight and branched C₁-C₆alkyl, unless otherwise noted. Examples of suitable straight and branched C₁-C₆alkyl substituents include methyl, ethyl, n-propyl, 2-propyl, n-butyl, sec-butyl, t-butyl, and the like. Unless otherwise noted, the alkyl substituents include both unsubstituted alkyl groups and alkyl groups that are substituted by one or more suitable substituents, including unsaturation (i.e. there are one or more double or triple C—C bonds), acyl, cycloalkyl, halo, oxyalkyl, alkylamino, aminoalkyl, acylamino and OR₁₅, for example, alkoxy. Preferred substituents for alkyl groups include halo, hydroxy, alkoxy, oxyalkyl, alkylamino, and aminoalkyl.

Cycloalkyl substituents include C₃-C₉ cycloalkyl groups, such as cyclopropyl, cyclobutyl, cyclopentyl, cyclohexyl and the like, unless otherwise specified. Unless otherwise noted, cycloalkyl substituents include both unsubstituted cycloalkyl groups and cycloalkyl groups that are substituted by one or more suitable substituents, including C₁-C₆ alkyl, halo, hydroxy, aminoalkyl, oxyalkyl, alkylamino, and OR₁₅, such as alkoxy. Preferred substituents for cycloalkyl groups include halo, hydroxy, alkoxy, oxyalkyl, alkylamino and aminoalkyl.

The above discussion of alkyl and cycloalkyl substituents also applies to the alkyl portions of other substituents, such as without limitation, alkoxy, alkyl amines, alkyl ketones, arylalkyl, heteroarylalkyl, alkylsulfonyl and alkyl ester substituents and the like.

Heterocycloalkyl substituents include 3 to 9 membered aliphatic rings, such as 4 to 7 membered aliphatic rings, containing from one to three heteroatoms selected from nitrogen, sulfur, oxygen. Examples of suitable heterocycloalkyl substituents include pyrrolidyl, tetrahydrofuryl, tetrahydrothiofuranyl, piperidyl, piperazyl, tetrahydropyranyl, morphilino, 1,3-diazapane, 1,4-diazapane, 1,4-oxazepane, and 1,4-oxathiapane. Unless otherwise noted, the rings are unsubstituted or substituted on the carbon atoms by one or more suitable substituents, including C₁-C₆ alkyl, C₄-C₉ cycloalkyl, aryl, heteroaryl, arylalkyl (e.g., benzyl), and heteroarylalkyl (e.g., pyridylmethyl), halo, amino, alkyl amino and OR₁₅, for example alkoxy. Unless otherwise noted, nitrogen heteroatoms are unsubstituted or substituted by H, C₁-C₄ alkyl, arylalkyl (e.g., benzyl), and heteroarylalkyl (e.g., pyridylmethyl), acyl, aminoacyl, alkylsulfonyl, and arylsulfonyl.

Cycloalkylalkyl substituents include compounds of the formula —(CH₂)ₙ₅-cycloalkyl wherein n5 is a number from 1-6. Suitable alkylcycloalkyl substituents include cyclopentylmethyl-, cyclopentylethyl, cyclohexylmethyl and the like. Such substituents are unsubstituted or substituted in the alkyl portion or in the cycloalkyl portion by a suitable substituent, including those listed above for alkyl and cycloalkyl.

Aryl substituents include unsubstituted phenyl and phenyl substituted by one or more suitable substituents, including C₁-C₆ alkyl, cycloalkylalkyl (e.g., cyclopropylmethyl), O(CO)alkyl, oxyalkyl, halo, nitro, amino, alkylamino, aminoalkyl, alkyl ketones, nitrile, carboxyalkyl, alkylsulfonyl, aminosulfonyl, arylsulfonyl, and OR₁₅, such as alkoxy. Preferred substituents include including C₁-C₆ alkyl, cycloalkyl (e.g., cyclopropylmethyl), alkoxy, oxyalkyl, halo, nitro, amino, alkylamino, aminoalkyl, alkyl ketones, nitrile, carboxyalkyl, alkylsulfonyl, arylsulfonyl, and aminosulfonyl. Examples of suitable aryl groups include C₁-C₄alkylphenyl, C₁-C₄alkoxyphenyl, trifluoromethylphenyl, methoxyphenyl, hydroxyethylphenyl, dimethylaminophenyl, aminopropylphenyl, carbethoxyphenyl, methanesulfonylphenyl and tolylsulfonylphenyl.

Aromatic polycycles include naphthyl, and naphthyl substituted by one or more suitable substituents, including C₁-C₆ alkyl, alkylcycloalkyl (e.g., cyclopropylmethyl), oxyalkyl, halo, nitro, amino, alkylamino, aminoalkyl, alkyl ketones, nitrile, carboxyalkyl, alkylsulfonyl, arylsulfonyl, aminosulfonyl and OR₁₅, such as alkoxy.

Heteroaryl substituents include compounds with a 5 to 7 member aromatic ring containing one or more heteroatoms, for example from 1 to 4 heteroatoms, selected from N, O and S. Typical heteroaryl substituents include furyl, thienyl, pyrrole, pyrazole, triazole, thiazole, oxazole, pyridine, pyrimidine, isoxazolyl, pyrazine and the like. Unless otherwise noted, heteroaryl substituents are unsubstituted or substituted on a carbon atom by one or more suitable substituents, including alkyl, the alkyl substituents identified above, and another heteroaryl substituent. Nitrogen atoms are unsubstituted or substituted, for example by R₁₃; especially useful N substituents include H, C₁-C₄ alkyl, acyl, aminoacyl, and sulfonyl.

Arylalkyl substituents include groups of the formula —(CH₂)ₙ₅-aryl, —(CH₂)ₙ₅₋₁—(CHaryl)-(CH₂)ₙ₅-aryl or —(CH₂)ₙ₅₋₁CH(aryl)(aryl) wherein aryl and n5 are defined above. Such arylalkyl substituents include benzyl, 2-phenylethyl, 1-phenylethyl, tolyl-3-propyl, 2-phenylpropyl, diphenylmethyl, 2-diphenylethyl, 5,5-dimethyl-3-phenylpentyl and the like. Arylalkyl substituents are unsubstituted or substituted in the alkyl moiety or the aryl moiety or both as described above for alkyl and aryl substituents.

Heteroarylalkyl substituents include groups of the formula —(CH₂)ₙ₅-heteroaryl wherein heteroaryl and n5 are defined above and the bridging group is linked to a carbon or a nitrogen of the heteroaryl portion, such as 2-, 3- or 4-pyridylmethyl, imidazolylmethyl, quinolylethyl, and pyrrolylbutyl. Heteroaryl substituents are unsubstituted or substituted as discussed above for heteroaryl and alkyl substituents.

Amino acyl substituents include groups of the formula —C(O)—(CH₂)ₙ—C(H)(NR₁₃R₁₄)—(CH₂)ₙ—R₅ wherein n, R₁₃, R₁₄ and R₅ are described above. Suitable aminoacyl substituents include natural and non-natural amino acids such as glycinyl, D-tryptophanyl, L-lysinyl, D- or L-homoserinyl, 4-aminobutryic acyl, ±3-amin-4-hexenoyl.

Non-aromatic polycycle substituents include bicyclic and tricyclic fused ring systems where each ring can be 4-9 membered and each ring can contain zero, 1 or more double and/or triple bonds. Suitable examples of non-aromatic polycycles include decalin, octahydroindene, perhydrobenzocycloheptene, perhydrobenzo-[f]-azulene. Such substituents are unsubstituted or substituted as described above for cycloalkyl groups.

Mixed aryl and non-aryl polycycle substituents include bicyclic and tricyclic fused ring systems where each ring can be 4-9 membered and at least one ring is aromatic. Suitable examples of mixed aryl and non-aryl polycycles include methylenedioxyphenyl, bis-methylenedioxyphenyl, 1,2,3,4-tetrahydronaphthalene, dibenzosuberane, dihydroanthracene, 9H-fluorene. Such substituents are unsubstituted or substituted by nitro or as described above for cycloalkyl groups.

Polyheteroaryl substituents include bicyclic and tricyclic fused ring systems where each ring can independently be 5 or 6 membered and contain one or more heteroatom, for example, 1, 2, 3, or 4 heteroatoms, chosen from O, N or S such that the fused ring system is aromatic. Suitable examples of polyheteroaryl ring systems include quinoline, isoquinoline, pyridopyrazine, pyrrolopyridine, furopyridine, indole, benzofuran, benzothiofuran, benzindole, benzoxazole, pyrroloquinoline, and the like. Unless otherwise noted, polyheteroaryl substituents are unsubstituted or substituted on a carbon atom by one or more suitable substituents, including alkyl, the alkyl substituents identified above and a substituent of the formula —O—(CH₂CH═CH(CH₃)(CH₂))₁₋₃H. Nitrogen atoms are unsubstituted or substituted, for example by R₁₃; especially useful N substituents include H, C₁-C₄ alkyl, acyl, aminoacyl, and sulfonyl.

Non-aromatic polyheterocyclic substituents include bicyclic and tricyclic fused ring systems where each ring can be 4-9 membered, contain one or more heteroatom, for example, 1, 2, 3, or 4 heteroatoms, chosen from O, N or S and contain zero or one or more C—C double or triple bonds. Suitable examples of non-aromatic polyheterocycles include hexitol, cis-perhydro-cyclohepta[b]pyridinyl, decahydro-benzo[f][1,4]oxazepinyl, 2,8-dioxabicyclo[3.3.0]octane, hexahydro-thieno[3,2-b]thiophene, perhydropyrrolo[3,2-b]pyrrole, perhydronaphthyridine, perhydro-1H-dicyclopenta[b,e]pyran. Unless otherwise noted, non-aromatic polyheterocyclic substituents are unsubstituted or substituted on a carbon atom by one or more substituents, including alkyl and the alkyl substituents identified above. Nitrogen atoms are unsubstituted or substituted, for example, by R₁₃; especially useful N substituents include H, C₁-C₄ alkyl, acyl, aminoacyl, and sulfonyl.

Mixed aryl and non-aryl polyheterocycles substituents include bicyclic and tricyclic fused ring systems where each ring can be 4-9 membered, contain one or more heteroatom chosen from O, N or S, and at least one of the rings must be aromatic. Suitable examples of mixed aryl and non-aryl polyheterocycles include 2,3-dihydroindole, 1,2,3,4-tetrahydroquinoline, 5,11-dihydro-10H-dibenz[b,e][1,4]diazepine, 5H-dibenzo[b,e][1,4]diazepine, 1,2-dihydropyrrolo[3,4-b][1,5]benzodiazepine, 1,5-dihydro-pyrido[2,3-b][1,4]diazepin-4-one, 1,2,3,4,6,11-hexahydro-benzo[b]pyrido[2,3-e][1,4]diazepin-5-one. Unless otherwise noted, mixed aryl and non-aryl polyheterocyclic substituents are unsubstituted or substituted on a carbon atom by one or more suitable substituents, including, —N—OH, ═N—OH, alkyl and the alkyl substituents identified above. Nitrogen atoms are unsubstituted or substituted, for example, by R₁₃; especially useful N substituents include H, C₁-C₄ alkyl, acyl, aminoacyl, and sulfonyl.

Amino substituents include primary, secondary and tertiary amines and in salt form, quaternary amines. Examples of amino substituents include mono- and di-alkylamino, mono- and di-aryl amino, mono- and di-arylalkyl amino, aryl-arylalkylamino, alkyl-arylamino, alkyl-arylalkylamino and the like.

Sulfonyl substituents include alkylsulfonyl and arylsulfonyl, for example methane sulfonyl, benzene sulfonyl, tosyl and the like.

Acyl substituents include groups of formula —C(O)—W, —OC(O)—W, —C(O)—O—W or —C(O)NR₁₃R₁₄, where W is R₁₆, H or cycloalkylalkyl.

Acylamino substituents include substituents of the formula —N(R₁₂)C(O)—W, —N(R₁₂)C(O)—O—W, and —N(R₁₂)C(O)—NHOH and R₁₂ and W are defined above.

The R₂ substituent HON—C(O)—CH═C(R₁)-aryl-alkyl- is a group of the formula

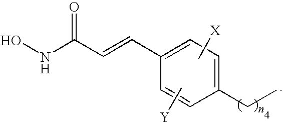

Preferences for each of the substituents include the following:
R₁ is H, halo, or a straight chain C₁-C₄ alkyl;
R₂ is selected from H, C₁-C₆ alkyl, C₄-C₉ cycloalkyl, C₄-C₉ heterocycloalkyl, alkylcycloalkyl, aryl, heteroaryl, arylalkyl, heteroarylalkyl, —(CH₂)ₙC(O)R₆, amino acyl, and —(CH₂)ₙR₇;
R₃ and R₄ are the same or different and independently selected from H, and C₁-C₆ alkyl, or R₃ and R₄ together with the carbon to which they are bound represent C═O, C═S, or C═NR₈;
R₅ is selected from H, C₁-C₆ alkyl, C₄-C₉ cycloalkyl, C₄-C₉ heterocycloalkyl, aryl, heteroaryl, arylalkyl, heteroarylalkyl, a aromatic polycycle, a non-aromatic polycycle, a mixed aryl and non-aryl polycycle, polyheteroaryl, a non-aromatic polyheterocycle, and a mixed aryl and non-aryl polyheterocycle;
n, n₁, n₂ and n₃ are the same or different and independently selected from 0-6, when n₁ is 1-6, each carbon atom is unsubstituted or independently substituted with R₃ and/or R₄;
X and Y are the same or different and independently selected from H, halo, C₁-C₄ alkyl, CF₃, NO₂, C(O)R₁, OR₉, SR₉, CN, and NR₁₀R₁₁;
R₆ is selected from H, C₁-C₆ alkyl, C₄-C₉ cycloalkyl, C₄-C₉ heterocycloalkyl, alkylcycloalkyl, aryl, heteroaryl, arylalkyl, heteroarylalkyl, OR₁₂, and NR₁₃R₁₄;
R₇ is selected from OR₁₅, SR₁₅, S(O)R₁₆, SO₂R₁₇, NR₁₃R₁₄, and NR₁₂SO₂R₆;
R₈ is selected from H, OR₁₅, NR₁₃R₁₄, C₁-C₆ alkyl, C₄-C₉ cycloalkyl, C₄-C₉ heterocycloalkyl, aryl, heteroaryl, arylalkyl, and heteroarylalkyl;
R₉ is selected from C₁-C₄ alkyl and C(O)-alkyl;
R₁₀ and R₁₁ are the same or different and independently selected from H, C₁-C₄ alkyl, and —C(O)-alkyl;
R₁₂ is selected from H, C₁-C₆ alkyl, C₄-C₉ cycloalkyl, C₄-C₉ heterocycloalkyl, aryl, heteroaryl, arylalkyl, and heteroarylalkyl;
R₁₃ and R₁₄ are the same or different and independently selected from H, C₁-C₆ alkyl, C₄-C₉ cycloalkyl, C₄-C₉ heterocycloalkyl, aryl, heteroaryl, arylalkyl, heteroarylalkyl and amino acyl;
R₁₅ is selected from H, C₁-C₆ alkyl, C₄-C₉ cycloalkyl, C₄-C₉ heterocycloalkyl, aryl, heteroaryl, arylalkyl, heteroarylalkyl and (CH₂)ₘZR₁₂;
R₁₆ is selected from C₁-C₆ alkyl, C₄-C₉ cycloalkyl, C₄-C₉ heterocycloalkyl, aryl, heteroaryl, arylalkyl, heteroarylalkyl and (CH₂)ₘZR₁₂;
R₁₇ is selected from C₁-C₆ alkyl, C₄-C₉ cycloalkyl, C₄-C₉ heterocycloalkyl, aryl, heteroaryl, arylalkyl, heteroarylalkyl and NR₁₃R₁₄;
m is an integer selected from 0 to 6; and
Z is selected from O, NR₁₃, S, S(O),
   or a pharmaceutically acceptable salt thereof.

Useful compounds of the formula (I) include those wherein each of R₁, X, Y, R₃, and R₄ is H, including those wherein one of n₂ and n₃ is zero and the other is 1, especially those wherein R₂ is H or —CH₂—CH₂—OH.

One suitable genus of hydroxamate compounds are those of formula Ia:

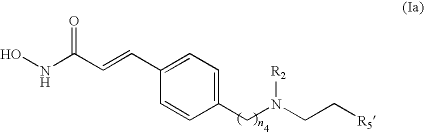

wherein
n₄ is 0-3,
R₂ is selected from H, C₁-C₆ alkyl, C₄-C₉ cycloalkyl, C₄-C₉ heterocycloalkyl, alkylcycloalkyl, aryl, heteroaryl, arylalkyl, heteroarylalkyl, —(CH₂)ₙC(O)R₆, amino acyl and —(CH₂)ₙR₇;
R₅′ is heteroaryl, heteroarylalkyl (e.g., pyridylmethyl), aromatic polycycles, non-aromatic polycycles, mixed aryl and non-aryl polycycles, polyheteroaryl, or mixed aryl and non-aryl polyheterocycles,
   or a pharmaceutically acceptable salt thereof.

Another suitable genus of hydroxamate compounds are those of formula Ia:

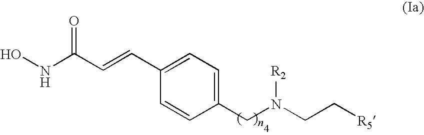

wherein
n₄ is 0-3,
R₂ is selected from H, C₁-C₆ alkyl, C₄-C₉ cycloalkyl, C₄-C₉ heterocycloalkyl, alkylcycloalkyl, aryl, heteroaryl, arylalkyl, heteroarylalkyl, —(CH₂)ₙC(O)R₆, amino acyl and —(CH₂)ₙR₇;
R₅′ is aryl, arylalkyl, aromatic polycycles, non-aromatic polycycles, and mixed aryl and non-aryl polycycles; especially aryl, such as p-fluorophenyl, p-chlorophenyl, p-O—C₁-C₄-alkylphenyl, such as p-methoxyphenyl, and p-C₁-C₄-alkylphenyl; and arylalkyl, such as benzyl, ortho, meta or para-fluorobenzyl, ortho, meta or para-chlorobenzyl, ortho, meta or para-mono, di or tri-O—C₁-C₄-alkylbenzyl, such as ortho, meta or para-methoxybenzyl, m,p-diethoxybenzyl, o,m,p-triimethoxybenzyl, and ortho, meta or para-mono, di or tri C₁-C₄-alkylphenyl, such as p-methyl, m,m-diethylphenyl,
   or a pharmaceutically acceptable salt thereof.

Another interesting genus are the compounds of formula Ib:

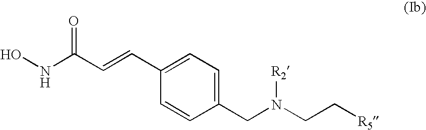

wherein
R₂′ is selected from H, C₁-C₆ alkyl, C₄-C₆ cycloalkyl, cycloalkylalkyl (e.g., cyclopropylmethyl), (CH₂)₂₋₄OR₂₁ where R₂₁ is H, methyl, ethyl, propyl, and i-propyl, and
R₅″ is unsubstituted 1H-indol-3-yl, benzofuran-3-yl or quinolin-3-yl, or substituted 1H-indol-3-yl, such as 5-fluoro-1H-indol-3-yl or 5-methoxy-1H-indol-3-yl, benzofuran-3-yl or quinolin-3-yl,
   or a pharmaceutically acceptable salt thereof.

Another interesting genus of hydroxamate compounds are the compounds of formula

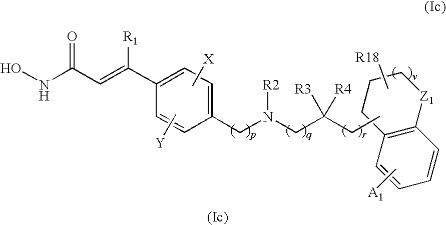

wherein
the ring containing Z₁ is aromatic or non-aromatic, which non-aromatic rings are saturated or unsaturated,
Z₁ is O, S or N—R₂₀,
R18 is H, halo, C₁-C₆alkyl (methyl, ethyl, t-butyl), C₃-C₇cycloalkyl, aryl, for example unsubstituted phenyl or phenyl substituted by 4-OCH₃ or 4-CF₃, or heteroaryl, such as 2-furanyl, 2-thiophenyl or 2-, 3- or 4-pyridyl;
R₂₀ is H, C₁-C₆alkyl, C₁-C₆alkyl-C₃-C₉cycloalkyl (e.g., cyclopropylmethyl), aryl, heteroaryl, arylalkyl (e.g., benzyl), heteroarylalkyl (e.g., pyridylmethyl), acyl (acetyl, propionyl, benzoyl) or sulfonyl (methanesulfonyl, ethanesulfonyl, benzenesulfonyl, toluenesulfonyl)
A₁ is 1, 2 or 3 substituents which are independently H, C₁-C-₆alkyl, —OR₁₉, halo, alkylamino, aminoalkyl, halo, or heteroarylalkyl (e.g., pyridylmethyl),
R₁₉ is selected from H, C₁-C₆alkyl, C₄-C₉cycloalkyl, C₄-C₉heterocycloalkyl, aryl, heteroaryl, arylalkyl (e.g., benzyl), heteroarylalkyl (e.g., pyridylmethyl) and —(CH₂CH═CH(CH₃)(CH₂))₁₋₃H;
R₂ is selected from H, C₁-C₆ alkyl, C₄-C₉ cycloalkyl, C₄-C₉ heterocycloalkyl, alkylcycloalkyl, aryl, heteroaryl, arylalkyl, heteroarylalkyl, —(CH₂)ₙC(O)R₆, amino acyl and —(CH₂)ₙR₇;
v is 0, 1 or 2,
p is 0-3, and
q is 1-5 and r is 0 or
q is 0 and r is 1-5,
   or a pharmaceutically acceptable salt thereof. The other variable substituents are as defined above.

Especially useful compounds of formula (Ic) are those wherein R₂ is H, or —(CH₂)ₚCH₂OH, wherein p is 1-3, especially those wherein R₁ is H; such as those wherein R₁ is H and X and Y are each H, and wherein q is 1-3 and r is 0 or wherein q is 0 and r is 1-3, especially those wherein Z₁ is N—R₂₀. Among these compounds R₂ is preferably H or —CH₂—CH₂—OH and the sum of q and r is preferably 1.

Another interesting genus of hydroxamate compounds are the compounds of formula (Id)

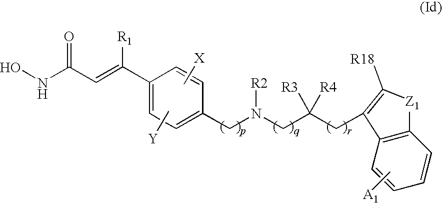

wherein

### Z₁ is O, S or N—R₂₀,

R18 is H, halo, C₁-C₆alkyl (methyl, ethyl, t-butyl), C₃-C₇cycloalkyl, aryl, for example, unsubstituted phenyl or phenyl substituted by 4-OCH₃ or 4-CF₃, or heteroaryl,
R₂₀ is H, C₁-C₆alkyl, C₁-C₆alkyl-C₃-C₉cycloalkyl (e.g., cyclopropylmethyl), aryl, heteroaryl, arylalkyl (e.g., benzyl), heteroarylalkyl (e.g., pyridylmethyl), acyl (acetyl, propionyl, benzoyl) or sulfonyl (methanesulfonyl, ethanesulfonyl, benzenesulfonyl, toluenesulfonyl),
A₁ is 1, 2 or 3 substituents which are independently H, C₁-C-₆alkyl, —OR₁₉, or halo,
R₁₉ is selected from H, C₁-C₆alkyl, C₄-C₉cycloalkyl, C₄-C₉heterocycloalkyl, aryl, heteroaryl, arylalkyl (e.g., benzyl), and heteroarylalkyl (e.g., pyridylmethyl);
p is 0-3, and
q is 1-5 and r is 0 or
q is 0 and r is 1-5,
or a pharmaceutically acceptable salt thereof. The other variable substituents are as defined above.

Especially useful compounds of formula (Id) are those wherein R₂ is H, or —(CH₂)ₚCH₂OH, wherein p is 1-3, especially those wherein R₁ is H; such as those wherein R₁ is H and X and Y are each H, and wherein q is 1-3 and r is 0 or wherein q is 0 and r is 1-3. Among these compounds R₂ is preferably H or CH₂—CH₂—OH and the sum of q and r is preferably 1.

The present invention further relates to compounds of the formula (Ie)

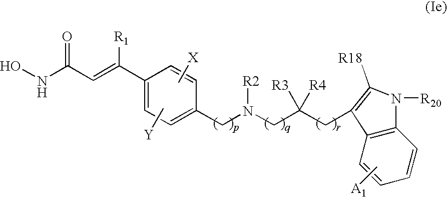

or a pharmaceutically acceptable salt thereof. The variable substituents are as defined above.

Especially useful compounds of formula (Ie) are those wherein R18 is H, fluoro, chloro, bromo, a C₁-C₄alkyl group, a substituted C₁-C₄alkyl group, a C₃-C₇cycloalkyl group, unsubstituted phenyl, phenyl substituted in the para position, or a heteroaryl (e.g., pyridyl) ring.

Another group of useful compounds of formula (Ie) are those wherein R₂ is H, or —(CH₂)ₚCH₂OH, wherein p is 1-3, especially those wherein R₁ is H; such as those wherein R₁ is H and X and Y are each H, and wherein q is 1-3 and r is 0 or wherein q is 0 and r is 1-3. Among these compounds R₂ is preferably H or —CH₂—CH₂—OH and the sum of q and r is preferably 1.

Another group of useful compounds of formula (Ie) are those wherein R18 is H, methyl, ethyl, t-butyl, trifluoromethyl, cyclohexyl, phenyl, 4-methoxyphenyl, 4 trifluoromethylphenyl, 2-furanyl, 2-thiophenyl, or 2-, 3- or 4-pyridyl wherein the 2-furanyl, 2-thiophenyl and 2-, 3- or 4-pyridyl substituents are unsubstituted or substituted as described above for heteroaryl rings; R₂ is H, or —(CH₂)ₚCH₂OH, wherein p is 1-3; especially those wherein R₁ is H and X and Y are each H, and wherein q is 1-3 and r is 0 or wherein q is 0 and r is 1-3. Among these compounds R₂ is preferably H or —CH₂—CH₂—OH and the sum of q and r is preferably 1.

Those compounds of formula Ie wherein R₂₀ is H or C₁-C₆alkyl, especially H, are important members of each of the subgenuses of compounds of formula Ie described above.

N-hydroxy-3-[4-[[(2-hydroxyethyl)[2-(1H-indol-3-yl)ethyl]-amino]methyl]phenyl]-2E-2-propenamide, N-hydroxy-3-[4-[[[2-(1H-indol-3-yl)ethyl]-amino]methyl]phenyl]-2E-2-propenamide and N-hydroxy-3-[4-[[[2-(2-methyl-1H-indol-3-yl)-ethyl]-amino]methyl]phenyl]-2E-2-propenamide, or a pharmaceutically acceptable salt thereof, are important compounds of formula (Ie).

Suitable are also compounds of the formula (If):

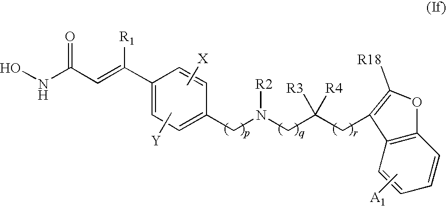

or a pharmaceutically acceptable salt thereof. The variable substituents are as defined above.

Useful compounds of formula (If) are include those wherein R₂ is H, or —(CH₂)ₚCH₂OH, wherein p is 1-3, especially those wherein R₁ is H; such as those wherein R₁ is H and X and Y are each H, and wherein q is 1-3 and r is 0 or wherein q is 0 and r is 1-3. Among these compounds R₂ is preferably H or —CH₂—CH₂—OH and the sum of q and r is preferably 1.

N-hydroxy-3-[4-[[[2-(benzofur-3-yl)-ethyl]-amino]methyl]phenyl]-2E-2-propenamide or a pharmaceutically acceptable salt thereof, is an important compound of formula (If).

Two preferred compounds within the scope of WO 02/22577 are N-hydroxy-3-[4-[(2-hydroxyethyl){2-(1H-indol-3-yl)ethyl]-amino]methyl]phenyl]-2E-2-propenamide, of formula (II) or a pharmaceutically acceptable salt thereof

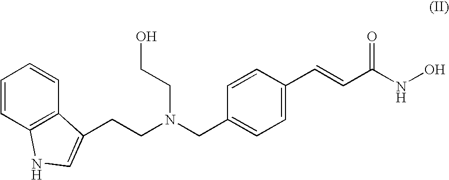

and N-hydroxy-3-[4-[[[2-(2-methyl-1H-indol-3-yl)-ethyl]-amino]methyl]phenyl]-2E-2-propenamide, of formula (III) below or a pharmaceutically acceptable salt thereof

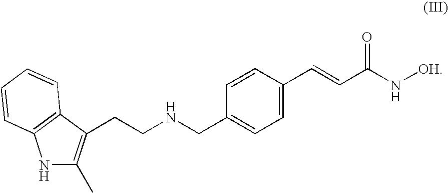

Most preferred examples of HDAC inhibitors are selected from the group consisting of MGCD-0103, MS27275, tacedinaline and compounds of formula (I), in particular N-hydroxy-3-[4-[(2-hydroxyethyl){2-(1H-indol-3-yl)ethyl]-amino]methyl]phenyl]-2E-2-propenamide or N-hydroxy-3-[4-[[[2-(2-methyl-1H-indol-3-yl)-ethyl]-amino]methyl]phenyl]-2E-2-propenamide, or a pharmaceutically acceptable salt thereof.

The combination of the present invention may comprise in addition (iii) a diuretic or a pharmaceutically acceptable salt thereof. A diuretic is, for example, a thiazide derivative selected from the group consisting of chlorothiazide, hydrochlorothiazide, methylclothiazide, and chlorothalidon. The most preferred diuretic is hydrochlorothiazide. A diuretic furthermore is a potassium sparing diuretic such as amiloride or triameterine, or a pharmaceutically acceptable salt thereof.

As indicated herein above, the compounds to be combined may be present as their pharmaceutically acceptable salts. If these compounds have, e.g., at least one basic center such as an amino group, they can form acid addition salts thereof. Similarly, the compounds having at least one acid group (for example COOH) can form salts with bases. Corresponding internal salts may furthermore be formed, if a compound comprises, e.g., both a carboxy and an amino group.

The corresponding active ingredients or a pharmaceutically acceptable salts may also be used in form of a solvate, such as a hydrate or including other solvents used, e.g., in their crystallization.

Preferred is a combination according to the present invention comprising (i) an angiotensin II blocker, e.g., valsartan, or a pharmaceutically acceptable salt thereof; and (ii) a HDAC inhibitor, e.g., N-hydroxy-3-[4-[(2-hydroxyethyl){2-(1H-indol-3-yl)ethyl]-amino]methyl]phenyl]-2E-2-propenamide or N-hydroxy-3-[4-[[[2-(2-methyl-1H-indol-3-yl)-ethyl]-amino]methyl]phenyl]-2E-2-propenamide, or a pharmaceutically acceptable salt thereof.

Preferred is also a combination according to the present invention comprising (i) an angiotensin II blocker, e.g., valsartan, or a pharmaceutically acceptable salt thereof; (ii) a HDAC inhibitor, e.g., N-hydroxy-3-[4-[(2-hydroxyethyl){2-(1H-indol-3-yl)ethyl]-amino]methyl]phenyl]-2E-2-propenamide or N-hydroxy-3-[4-[[[2-(2-methyl-1H-indol-3-yl)-ethyl]-amino]methyl]phenyl]-2E-2-propenamide, or a pharmaceutically acceptable salt thereof; and (iii) a diuretic, e.g., hydrochlorothiazide.

Furthermore, the present invention provides pharmaceutical compositions comprising:
(i) an angiotensin receptor blocker (ARB) or a pharmaceutically acceptable salt thereof, and
(ii) a histone deacetylase (HDAC) inhibitor or a pharmaceutically acceptable salt thereof;
and a pharmaceutically acceptable carrier.

As disclosed herein above, (i) an angiotensin II blocker, e.g., valsartan, or a pharmaceutically acceptable salt thereof; (ii) a HDAC inhibitor, e.g., N-hydroxy-3-[4-[(2-hydroxyethyl){2-(1H-indol-3-yl)ethyl]-amino]methyl]phenyl]-2E-2-propenamide or N-hydroxy-3-[4-[[[2-(2-methyl-1H-indol-3-yl)-ethyl]-amino]methyl]phenyl]-2E-2-propenamide, or a pharmaceutically acceptable salt thereof; and optionally (iii) a diuretic, e.g., hydrochlorothiazide, may be co-administered as a pharmaceutical composition. The components may be administered together in any conventional dosage form, usually also together with a pharmaceutically acceptable carrier or diluent.

The pharmaceutical compositions according to the invention are those suitable for enteral, such as oral or rectal, transdermal and parenteral administration to mammals, including man. For oral administration the pharmaceutical composition comprising an (i) an angiotensin II blocker, e.g., valsartan, or a pharmaceutically acceptable salt thereof; (ii) a HDAC inhibitor, e.g., N-hydroxy-3-[4-[(2-hydroxyethyl){2-(1H-indol-3-yl)ethyl]-amino]methyl]phenyl]-2E-2-propenamide or N-hydroxy-3-[4-[[[2-(2-methyl-1H-indol-3-yl)-ethyl]-amino]methyl]phenyl]-2E-2-propenamide, or a pharmaceutically acceptable salt thereof; and optionally (iii) a diuretic, e.g., hydrochlorothiazide, can take the form of solutions, suspensions, tablets, pills, capsules, powders, microemulsions, unit dose packets and the like. Preferred are tablets and gelatin capsules comprising the active ingredient together with: a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets also c) binders, e.g., magnesium aluminum silicate, starch paste, gelatin, tragacanth, methylcellulose, sodium carboxymethylcellulose and or polyvinylpyrrolidone; if desired d) disintegrants, e.g., starches, agar, alginic acid or its sodium salt, or effervescent mixtures; and/or e) absorbants, colorants, flavors and sweeteners. Injectable compositions are preferably aqueous isotonic solutions or suspensions, and suppositories are advantageously prepared from fatty emulsions or suspensions.

Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-90%, preferably about 1-80%, of the active ingredient.

The dosage of the active ingredients can depend on a variety of factors, such as mode of administration, homeothermic species, age and/or individual condition. Preferred dosages for the active ingredients of the combinations or pharmaceutical compositions according to the present invention are therapeutically effective dosages, especially those which are commercially available.

Normally, in the case of oral administration, an approximate daily dose of from about 1 mg to about 360 mg is to be estimated, e.g., for a patient of approximately 75 kg in weight. For example, angiotensin II receptor blockers, e.g., valsartan, are supplied in the form of a suitable dosage unit form, e.g., a capsule or tablet, and comprising a therapeutically effective amount of an angiotensin II receptor blocker, e.g., from about 20 to about 320 mg, of e.g. valsartan, which may be applied to patients. The application of the active ingredient may occur up to three times a day, starting, e.g., with a daily dose of 20 mg or 40 mg of an angiotensin II receptor blocker, e.g., valsartan, increasing via 80 mg daily and further to 160 mg daily, and finally up to 320 mg daily. Preferably, an angiotensin II receptor blocker, e.g., valsartan is applied once a day or twice a day with a dose of preferably 80 mg or 160 mg, respectively, each. Corresponding doses may be taken, e.g., in the morning, at mid-day or in the evening. Preferred is q.d. or b.i.d. administration in heart failure.

The doses of a HDAC inhibitor, e.g., N-hydroxy-3-[4-[(2-hydroxyethyl){(2-(1H-indol-3-yl)ethyl]-amino]methyl]phenyl]-2E-2-propenamide or N-hydroxy-3-[4-[[[2-(2-methyl-1H-indol-3-yl)-ethyl]-amino]methyl]phenyl]-2E-2-propenamide, or a pharmaceutically acceptable salt thereof, to be administered to warm-blooded animals, including man, of approximately 75 kg body weight, especially the doses effective for the inhibition of HDAC activity, e.g., in treating pathological, can be selected by the person skilled in the art. The HDAC inhibitor can be administered orally or intravenously. In case of diuretics, preferred dosage unit forms are, e.g., tablets or capsules comprising, e.g., from about 5 mg to about 200 mg, preferably, 5 mg to about 50 mg, more preferably 5 mg to about 25 mg, yet more preferably from about 6.25 mg to about 25 mg. In one embodiment 8 mg to about 16 mg is preferred. A daily dose of 6.25 mg, 12.5 mg or 25 mg of e.g. hydrochlorothiazide is preferably administered once a day.

The above doses encompass a therapeutically effective amount of the active ingredients of the present invention

Typical dosages for valsartan in drinking water range from 1 to 100 mg/kg/day, and dosages of HCTZ range from 1 to 75 mg/kg/day. In most situations, a daily dose will not exceed 100 mg/kg/day when administered as the monotherapy. In combination, lower dosages of each agent are used and correspondingly, valsartan is given in the range of 1 to 30 mg/kg/day, and HCTZ are give in dosages below 50 mg/kg/day.

When drugs are administered by oral gavage, the dose of valsartan ranges from 1 to 50 mg/kg/day and HCTZ does not exceed 75 mg/kg/day, respectively. An example of a preferred combination, comprises an amount of Valsartan from 20 to 640 mg.

Another example of a preferred combination, comprises an amount of Valsartan from 20 to 640 mg, and an amount of HCTZ of 8 to 16 mg. Another example of a preferred combination, comprises an amount of Valsartan from 40 to 320 mg.

Another example of a preferred combination, comprises an amount of Valsartan from 40 to 320 mg, and an amount of HCTZ of 8 to 16 mg.

Another example of a preferred composition, comprises an amount of Valsartan from 60 to 100 mg e.g. 80 mg.

Another example of a preferred composition, comprises an amount of Valsartan from 60 to 100 mg e.g. 80 mg, an amount of HCTZ from 8 to 16 mg, e.g. 12.5 mg.

Another example of a preferred composition, comprises an amount of Valsartan from 140 to 180 mg e.g. 160 mg.

Another example of a preferred composition, comprises an amount of Valsartan from 140 to 180 mg e.g. 160 mg, and an amount of HCTZ between 8 and 16 mg e.g. 12.5 mg.

The combination of (i) an ARB, (ii) a histone deacetylase (HDAC) inhibitor, and optionally (iii) a diuretic may, according to the present invention be manufactured and administered in free or fixed dose combinations of the respective pharmaceutically active agents. It may be advantageous to begin the treatment with free combinations that allow an easy adjustment of the administered dose of each individual agent. When the ideal dose regimen, which generally is dependent on the specific condition of the individual to be treated, the individuals weight, other medication administered to the individual and the like, is reached, a fixed dose combination may be administered in case where an administration once a day or e.g. twice or three times daily is possible and a sufficient control of blood pressure is achieved. Presently it is preferred to combine two of the components (i) to (iii) and administer the third separately at the same or at a different time.

Valsartan is being marketed under the trade name Diovan®. A combination of valsartan and HCTZ is being marketed under the trade name Co-Diovan®. All of these marketed products may be utilized in as such for combination therapy according to the present invention.

The invention also relates to combining separate pharmaceutical compositions in kit form. That is a kit combining two or three separate units: e.g. a pharmaceutical composition comprising an ARB and a pharmaceutical composition comprising a histone deacetylase (HDAC) inhibitor; or a pharmaceutical composition comprising an ARB, a pharmaceutical composition comprising a histone deacetylase (HDAC) inhibitor and a pharmaceutical composition comprising a diuretic. Although the kit form is particularly advantageous when the separate components must be administered in different dosage forms (e.g. parenteral valsartan formulation and oral hydrochlorothiazide formulations) or are administered at different dosage intervals, the administration of the single components of such a kit of parts may, without any restriction be effected simultaneously, sequentially or staggered with time.

In a preferred embodiment, the (commercial) product is a commercial package comprising as active ingredients the combination according to the present invention (in the form of two or three separate units of the components (i) and (ii) or (i) to (iii)), together with instructions for its simultaneous, separate or sequential use, or any combination thereof, in the delay of progression or treatment of the diseases mentioned herein. A preferred commercial package, is where the ARB (i) is present in the form of DIOVAN®. Another preferred commercial package, is where the ARB (i) and the diuretic (iii) are present in the form of Co-DIOVAN®.

The pharmaceutical preparations of the present invention are for enteral, such as oral, and also rectal or parenteral, administration to homeotherms, with the preparations comprising the pharmacological active compound either alone or together with customary pharmaceutical auxiliary substances. For example, the pharmaceutical preparations consist of from about 0.1% to 90%, preferably of from about 1% to about 80%, of the active compounds. Pharmaceutical preparations for enteral or parenteral administration are, for example, in unit dose forms, such as coated tablets, tablets, capsules or suppositories and also ampoules. These are prepared in a manner, which is known per se, for example using conventional mixing, granulation, coating, solubilizing or lyophilizing processes. Thus, pharmaceutical preparations for oral use can be obtained by combining the active compounds with solid excipients, if desired granulating a mixture which has been obtained, and, if required or necessary, processing the mixture or granulate into tablets or coated tablet cores after having added suitable auxiliary substances.

The dosage of the active compound can depend on a variety of factors, such as mode of administration, homeothermic species, age and/or individual condition. Preferred dosages for the active ingredients of the pharmaceutical combination according to the present invention are therapeutically effective dosages, especially those that are commercially available. Normally, in the case of oral administration, an approximate daily dose of from about 20 mg to about 900 mg of active agents, i.e. ARB plus histone deacetylase (HDAC) inhibitor or ARB plus histone deacetylase (HDAC) inhibitor plus diuretic, is to be estimated e.g. for a patient of approximately 75 kg in weight.

In the present invention preferred ARBs are those agents that have been marketed, as e.g. valsartan and losartan. In the present invention preferred histone deacetylase (HDAC) inhibitors are those agents that are currently developed, e.g. N-hydroxy-3-[4-[(2-hydroxyethyl){2-(1H-indol-3-yl)ethyl]-amino]methyl]phenyl]-2E-2-propenamide or N-hydroxy-3-[4-[[[2-(2-methyl-1H-indol-3-yl)-ethyl]-amino]methyl]phenyl]-2E-2-propenamide. The most preferred diuretic is hydrochlorothiazide (HCTZ).

Very surprisingly is the finding that, a combination of (i) an ARB, (ii) a histone deacetylase (HDAC) inhibitor, and optionally (iii) a diuretic and in particular a combination comprising valsartan and N-hydroxy-3-[4-[(2-hydroxyethyl){2-(1H-indol-3-yl)ethyl]-amino]methyl]phenyl]-2E-2-propenamide or N-hydroxy-3-[4-[[[2-(2-methyl-1H-indol-3-yl)-ethyl]-amino]methyl]phenyl]-2E-2-propenamide, or valsartan, N-hydroxy-3-[4-[(2-hydroxyethyl){2-(1H-indol-3-yl)ethyl]-amino]methyl]phenyl]-2E-2-propenamide or N-hydroxy-3-[4-[[[2-(2-methyl-1H-indol-3-yl)-ethyl]-amino]methyl]phenyl]-2E-2-propenamide, and HCTZ achieves greater therapeutic effect than the administration of the respective therapeutic agents alone. The combination of the present invention is therefore particularly useful in cases where the use of an ARB alone does not satisfactorily treat the respective disorder.

It has been surprisingly found that a combination of (i) an ARB, (ii) a histone deacetylase (HDAC) inhibitor, and optionally (iii) a diuretic improves left ventricle function, without increasing the myocardial oxygen requirement. Furthermore such a combination does not act directly to stimulate cardiac contractility, or produces side-effects such as changes in blood pressure and/or heart rate, which are associated with increased mortality in patients with HF. It has also been surprisingly found that a combination of (i) an ARB, (ii) a histone deacetylase (HDAC) inhibitor, and optionally (iii) a diuretic is particularly safe (non toxic) and useful for long-term administration e.g. less side effects, good absorbability into the body upon oral administration and long-lasting action.

In particular the combined administration of a combination of (i) an ARB, (ii) a histone deacetylase (HDAC) inhibitor, and optionally (iii) a diuretic results in a significant response in a greater percentage of treated patients compared to monotherapy, that is, a greater responder rate results, regardless of the underlying etiology of the condition. This is in accordance with the desires and requirements of the patients to be treated. The combination is also useful in the treatment or prevention of heart failure such as (acute and chronic) congestive heart failure, left ventricular dysfunction and hypertrophic cardiomyopathy, diabetic cardiac myopathy, supraventricular and ventricular arrhythmias, atrial fibrillation, atrial flutter or detrimental vascular remodeling. A physical combination of an Ang II receptor blocker (e.g valsartan) and an HDAC inhibitor acting in tandem at strategic nodal points along the biochemical pathways mediating pathological hypertrophy acts synergistically and ameliorates or even reverses established pathological hypertrophy and heart failure. It can further be shown that a combination therapy proves to be beneficial in the treatment and prevention of myocardial infarction and its sequelae. A combination is also useful in treating atherosclerosis, angina (whether stable or unstable), renal insufficiency (diabetic and non-diabetic), peripheral vascular disease, cognitive dysfunction, and stroke. Furthermore, the improvement in endothelial function with the combination therapy provides benefit in diseases in which normal endothelial function is disrupted such as heart failure, angina pectoris and diabetes. Furthermore, the combination of the present invention may be used for the treatment or prevention of secondary aldosteronism, primary and secondary pulmonary hypertension, renal failure conditions, such as diabetic nephropathy, glomerulonephritis, scleroderma, glomerular sclerosis, proteinuria of primary renal disease, and also renal vascular hypertension, diabetic retinopathy, the management of other vascular disorders, such as migraine, peripheral vascular disease, Raynaud's disease, luminal hyperplasia, cognitive dysfunction (such as Alzheimer's), glaucoma and stroke. The combination regimen also surprisingly reduces the rate of progression of cardiac, renal and cerebral end-organ damage. By providing enhanced efficacy, safety and tolerability, the combination of drugs indicated in this invention also has the potential to promote patient compliance, a major consideration in the pharmacological treatment of cardiovascular diseases.

The person skilled in the pertinent art is fully enabled to select a relevant test model to prove the efficacy of a combination of the present invention in the herein before and hereinafter indicated therapeutic indications.

The advantages of the present combinations are, for example, demonstrated in a clinical study or in the test procedure as essentially described hereinafter. Many clinical study protocols adapted to test our combinations are known by the person skilled in the art. Examples of models useful to demonstrate the unexpected advantages of our new combinations are described below.

Representative studies are carried out with a combination of valsartan, a suitable HDAC inhibitor, and HCTZ applying the following methodologies.

1. The ascending or transverse aortic-banded mouse models are used as pressure-overload models to ascertain the beneficial effects of the combination of an HDAC inhibitor and an ARB (e.g. valsartan) on pathological cardiac hypertrophy. The methods described by Tarnavski et al. (2004) or Ogita et al. (2004) are used for this purpose. Briefly, anesthetized C57BL/6 male mice (age, 11 to 12 weeks) are subjected to the surgical procedure of ascending or transverse aortic banding. Sham-operated mice are subjected to similar surgical procedures without constriction of the aorta.

Blood pressure and heart rate are measured non-invasively in conscious animals before and periodically after surgery by the tail-cuff plethysmography method. Under light anesthesia, 2-dimensional guided M-mode echocardiography is performed. The percentage of left ventricular fractional shortening is calculated as [(LVDD−LVSD)/LVDD]×100(%) as described by Ogita et al. (2004). LVDD and LVSD indicate left ventricular end-diastolic and end-systolic chamber dimensions, respectively. Left ventricular mass was calculated as 1.055[(LVDD+PWTD+VSTD)3−(LVDD)3] (mg), where PWTD indicates diastolic posterior wall thickness, and VSTD indicates diastolic ventricular septal thickness.

After the above assessments, the animals are randomly segregated into aortic-banding or sham-operated groups. At the end of the aortic-banding operation, the animals are assigned to either the control (vehicle-treated) group or to the test (drug-treated, singly or in combination) groups. All groups are followed for not less than 4 weeks before using them for data analysis.

Hearts are excised after the mice are euthanized with an overdose injection of an anesthetic. Ratios of heart weight to body weight are ascertained. Sections of the hearts are prepared as previously described by Tarnavski et al. (2004), stained with hematoxylin-eosin and Masson's trichrome and observed under light microscopy.

2. The beneficial effects of the combination of an HDAC inhibitor and an ARB (e.g. valsartan) on cardiac hypertrophy and heart failure are ascertained in a murine model of myocardial infarction and heart failure. Myocardial infarction is induced in mice (age, 11-12 weeks) by ligating the left anterior descending (LAD) coronary artery under anesthesia as described by Tarnavski et al. (2004). Sham operated animals undergo the same experimental procedures but without coronary ligation.

Blood pressure and heart rate are measured non-invasively in conscious animals before and periodically after surgery by the tail-cuff plethysmography method. Under light anesthesia, 2-dimensional guided M-mode echocardiography is performed. The percentage of LV fractional shortening is calculated as [(LVDD−LVSD)/LVDD]×100(%) as described by Ogita et al. (2004). LVDD and LVSD indicate left ventricular end-diastolic and end-systolic chamber dimensions, respectively. Left ventricular mass was calculated as 1.055[(LVDD+PWTD+VSTD)3-(LVDD)₃] (mg), where PWTD indicates diastolic posterior wall thickness, and VSTD indicates diastolic ventricular septal thickness.

An invasive method for blood pressure measurement is used prior to the animal sacrifice. A micromanometer tipped Millar catheter (1.4 French) is inserted into the right carotid artery and advanced into the LV chamber to measure LV pressure.

After the above assessments, the animals (ligated, sham operated) are segregated into indicated groups and treated with the test compounds (singly and in combination) or corresponding vehicles. All groups are followed for not less than 14 days before using them for data analysis.

Hearts are excised after the mice are euthanized with an overdose injection of an anesthetic. Ratios of heart weight to body weight are ascertained. Transverse sections of the hearts are prepared as previously described by Tarnavski et al. (2004), stained with hematoxylin-eosin and Masson's trichrome and observed under light microscopy.

3. The beneficial effects of a combination of an HDAC inhibitor and an ARB (e.g. valsartan) on cardiac hypertrophy induced by tachycardia in dogs are also ascertained. The techniques described by Motte et al. (2003) with minor modifications are used in these studies. Briefly, a bipolar pacemaker lead is surgically advanced through the right jugular vein and implanted in the right ventricular apex of anesthetized mongrel dogs. A programmable pulse generator is inserted into a subcuticular cervical pocket and connected to the pacemaker lead.

The animals undergo a pacing protocol with a stepwise increase of stimulation frequencies as described by Motte et al. (2003). Pacing is initiated by activating the pulse generator at 180 beats/min and continued for 1 week, followed by 200 beats/min over a second week, 220 beats/min over a third week, and finally 240 beats/min over the last 2 wk. The investigations are carried out at baseline (week 0) and once weekly throughout the pacing period (i.e., from week 1 to week 5). On the third day of pacing, the test agents (singly and in combination) or matching placebo is administered and continued on the same daily dose until the end of the study at five weeks.

Body weight, rectal temperature, heart rate (HR), respiratory rate (RR), and blood pressure is monitored. Doppler echocardiography is performed under continuous ECG monitoring with a 3.5- to 5-MHz mechanical sector probe. Left ventricular internal end-diastolic (LVIDd) and systolic diameters (LVIDs) as well as systolic and diastolic left ventricular free wall (LVFWs and LVFWd) and interventricular septum thickness (IVSs and IVSd) are determined. An image of the aortic flow is obtained by pulsed-wave Doppler. The velocity spectra are used to measure the preejection period (PEP) and left ventricular ejection time (LVET). From these data, left ventricular end-diastolic (EDV) and systolic volume (ESV), left ventricular ejection fraction (LVEF), and mean velocity of circumferential fiber shortening (MVCF) are calculated.

The following examples illustrate the invention described above and are not intended to restrict the scope of this invention in any way.

### FORMULATION EXAMPLE 1

### Composition and Batch Quantities for Diovan® Tablets

| | COMPOSITION PER UNIT | | | | | | | |
|---|---|---|---|---|---|---|---|---|
| Components | (mg) | | | | QUANTITY PER BATCH¹ (kg) | | | |
| Granulation | 40 mg | 80 mg | 160 mg | 320 mg | 40 mg | 80 mg | 160 mg | 320 mg |
| Diovan Drug | 40.000 | 80.000 | 160.000 | 320.000 | 144.000 | 144.000 | 144.000 | 144.000 |
| Substance | | | | | | | | |
| Microcrystalline | 27.000 | 54.000 | 108.000 | 216.000 | 97.200 | 97.200 | 97.200 | 97.200 |
| Cellulose(NF, Ph. Eur.) | | | | | | | | |
| Avicel PH102 | | | | | | | | |
| Crospovidone | 7.500 | 15.000 | 30.000 | 60.000 | 27.000 | 27.000 | 27.000 | 27.000 |
| (NF, Ph. Eur.) | | | | | | | | |
| Colloidal Anhydrous | 0.750 | 1.500 | 3.000 | 6.000 | 2.700 | 2.700 | 2.700 | 2.700 |
| Silica | | | | | | | | |
| (Ph. Eur.)/Colloidal | | | | | | | | |
| silicon Dioxide | | | | | | | | |
| (NF)/Aerosil 200 | | | | | | | | |
| Magnesium Stearate | 1.500 | 3.000 | 6.000 | 12.000 | 5.400 | 5.400 | 5.400 | 5.400 |
| (NF, Ph. Eur.) | | | | | | | | |
| Blending | | | | | | | | |
| Magnesium Stearate | 0.750 | 1.500 | 3.000 | 6.000 | 2.700 | 2.700 | 2.700 | 2.700 |
| (NF, Ph. Eur.) | | | | | | | | |
| Coating | | | | | | | | |
| DIOLACK Gelb | 2.800 | | | | 11.090² | | | |
| F32892 | | | | | | | | |
| DIOLACK Blassrot | | 6.000 | | | | 12.420³ | | |
| F34899 | | | | | | | | |
| DIOLACK Hellbraun | | | 9.000 | | | | 9.720⁴ | |
| F33172 | | | | | | | | |
| DIOLACK Braun | | | | 16.000 | | | | 8.640⁴ |
| F16711 | | | | | | | | |
| Purified Water | | | | | 62.843 | 70.380 | 55.080 | 48.960 |
| Total Tablet/Batch | 80.300 | 161.000 | 319.000 | 636.000 | 289.080 | 289.800 | 287.100 | 286.200 |
| Weight | | | | | | | | |

¹A total of 2 subdivisions of granulation per batch
²A 10% excess of coating solution was manufactured to account for loss during coating.
³A 15% excess of coating solution was manufactured to account for loss during coating.
⁴A 20% excess of coating solution was manufactured to account for loss during coating.

### Composition of Diolack

| | | | | Iron Oxide | Iron Oxide | Iron Oxide | Iron |
|---|---|---|---|---|---|---|---|
| | | | Titanium | (Red) | (Yellow) | (Brown) | Oxide |
| | | PEG | Dioxide | Ph. Fr./NF/ | Ph. Fr./NF/ | Mixture of | (Black) |
| | HPMC | 8000 | (White) | E172/CFR/ | E172/CFR/ | iron oxide | E172/CFR/ |
| | USP/Ph. Eur | USP/Ph. | USP/Ph. | CI | CI | red & | CI |
| DIOLACK | (603) | Eur. | Eur | 77491 | 77492 | black | 77499 |
| Gelb | 80.00% | 4.00% | 13.48% | 0.01% | 2.50% | — | 0.01% |
| F32892 | | | | | | | |
| Blassrot | 80.00% | 4.00% | 15.50% | 0.40% | 0.10% | — | — |
| F34899 | | | | | | | |
| Hellbraun | 80.00% | 4.00% | 9.34% | 0.25% | 6.40% | — | 0.01% |
| F33172 | | | | | | | |
| Braun | 80.00% | 4.00% | 14.00% | 0.50% | 0.50% | 0.50% | 0.50% |
| F16711 | | | | | | | |

A mixture of Diovan drug substance, microcrystalline cellulose, crospovidone, part of the colloidal anhydrous silica/colloidal silicon dioxide/Aerosile 200, silicon dioxide and magnesium stearate is premixed in a diffusion mixer and then sieved through a screening mill. The resulting mixture is again pre-mixed in a diffusion mixer, compacted in a roller compacter and then sieved through a screening mill. To the resulting mixture, the rest of the colloidal anhydrous silica/colloidal silicon dioxide/Aerosile 200 are added and the final blend is made in a diffusion mixer. The whole mixture is compressed in a rotary tabletting machine and the tablets are coated with a film by using the appropriate composition of Diolack in a perforated pan.

### FORMULATION EXAMPLE 2

### Composition and Quantities for Co-Diovan® Tablets

| | COMPOSITION | COMPOSITION | COMPOSITION |
|---|---|---|---|
| Components | PER UNIT (mg) | PER UNIT (mg) | PER UNIT (mg) |
| Granulation | | | |
| Diovan Drug | 80.000 | 160.000 | 160.00 |
| Substance | | | |
| Esidrex Drug | 12.500 | 12.500 | 25.00 |
| Substance (micro) | | | |
| Microcrystalline | 31.500 | 75.500 | 63.00 |
| Cellulose (NF, | | | |
| Ph. Eur.)/Avicel | | | |
| PH 102 | | | |
| Crospovidone | 20.000 | 40.000 | 40.00 |
| (NF, Ph. Eur.) | | | |
| Colloidal Anhydrous | 1.500 | 3.00 | 3.00 |
| Silica (Ph. Eur.)/ | | | |
| Colloidal Silicon | | | |
| Dioxide (NF)/ | | | |
| Aerosil 200 | | | |
| Magnesium Stearate | 3.000 | 6.000 | 6.00 |
| (NF, Ph. Eur.) | | | |
| Blending | | | |
| Magnesium Stearate, | 1.500 | 3.000 | 3.00 |
| NF, Ph. Eur. | | | |
| Coating | | | |
| Opadry Black | — | — | 0.096 |
| OOF17713 | | | |
| Opadry Red | — | — | 0.762 |
| OOF15613 | | | |
| Opadry Yellow | — | — | 3.808 |
| OOF12951 | | | |
| Opadry White | — | — | 5.334 |
| OOF18296 | | | |
| Hydroxy propyl | 2.76 | 5.510 | — |
| Methylcellulose | | | |
| Iron Oxide Yellow | 0.025 | — | — |
| Iron Oxide Red | 0.025 | 0.750 | — |
| Polyethylene | 0.50 | 1.000 | — |
| Glycol 8000 | | | |
| Talc | 2.000 | 3.990 | — |
| Titanium Dioxide | 0.70 | 0.750 | — |
| Total Tablet/ | 156.000 | 312.000 | 310.00 |
| Batch Weight | | | |

### Composition of Opadry

| | | | | | Iron | | |
|---|---|---|---|---|---|---|---|
| | | | | | Oxide | Iron Oxide | |
| | | | | Titanium | (Red) | (Yellow) | Iron Oxide |
| | HPMC | PEG | | Dioxide | Ph. Fr./NF/ | Ph. Fr./NF/ | (Black) |
| | USP/Ph. | 4000 | Talc | USP/Ph. | E172/CFR/ | E172/CFR/ | E172/CFR/ |
| | Eur | USP/Ph. | USP/Ph. | Eur | CI | CI | CI |
| OPADRY | (603) | Eur. | Eur | (White) | 77491 | 77492 | 77499 |
| Opadry | 71.4% | 7.15% | 7.15% | 14.3% | — | — | — |
| White | | | | | | | |
| OOF18296* | | | | | | | |
| Opadry | 71.4% | 7.15% | 7.15% | — | 14.3% | — | — |
| Red | | | | | | | |
| OOF15613* | | | | | | | |
| Opadry | 71.4% | 7.15% | 7.15% | — | — | 14.3% | — |
| Red | | | | | | | |
| OOF15613* | | | | | | | |
| Opadry | 71.4% | 7.15% | 7.15% | — | — | — | 14.3% |
| Black | | | | | | | |
| OOF17713* | | | | | | | |

A mixture of Diovan drug substance, Esidrex drug substance (micro), microcrystalline cellulose, crospovidone, colloidal anhydrous silica/Aerosil 200 and part of the magnesium stearate is premixed in a diffusion mixer and then sieve through a screening mill. The resulting mixture is again pre-mixed in a diffusion mixer, compacted in a roller compacter and then sieved through a screening mill. The final blend is made in a diffusion mixer under addition of the remaining part of the magnesium stearate, which is hand screened before.

The whole mixture is compressed in a rotary tabletting machine and the tablets are coated with a film by using the appropriate composition of Opadry in a perforated pan.

Subjecting the combination of valsartan and e.g. N-hydroxy-3-[4-[(2-hydroxyethyl){2-(1H-indol-3-yl)ethyl]-amino]methyl]phenyl]-2E-2-propenamide or N-hydroxy-3-[4-[[[2-(2-methyl-1H-indol-3-yl)-ethyl]-amino]methyl]phenyl]-2E-2-propenamide to the test models outlined above in 1 to 3 could demonstrate the suitability and advantages in the treatment of e.g. heart failure.

1. A combination comprising
(i) an angiotensin receptor blocker (ARB) or a pharmaceutically acceptable salt thereof, and
(ii) a histone deacetylase (HDAC) inhibitor or a pharmaceutically acceptable salt thereof.

2. A combination according to claim 1, wherein (i) the angiotensin receptor blocker (ARB) is selected from the group consisting of candesartan, eprosartan, irbesartan, losartan, olmesartan, saprisartan, tasosartan, telmisartan, valsartan, E-4177, SC-52458, and ZD8731; and (ii) the histone deacetylase (HDAC) inhibitor is selected from the group consisting of MGCD-0103, MS27275, tacedinaline and compounds of formula (I) 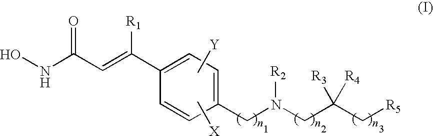
wherein
R₁ is H, halo, or a straight chain C₁-C₆ alkyl (especially methyl, ethyl or n-propyl, which methyl, ethyl and n-propyl substituents are unsubstituted or substituted by one or more substituents described below for alkyl substituents);
R₂ is selected from H, C₁-C₁₀ alkyl, (preferably C₁-C₆ alkyl, e.g. methyl, ethyl or —CH₂CH₂—OH), C₄-C₉ cycloalkyl, C₄-C₉ heterocycloalkyl, C₄-C₉ heterocycloalkylalkyl, cycloalkylalkyl (e.g., cyclopropylmethyl), aryl, heteroaryl, arylalkyl (e.g. benzyl), heteroarylalkyl (e.g. pyridylmethyl), —(CH₂)ₙC(O)R₆, —(CH₂)ₙOC(O)R₆, amino acyl, HON—C(O)—CH═C(R₁)-aryl-alkyl- and —(CH₂)ₙR₇;
R₃ and R₄ are the same or different and independently H, C₁-C₆ alkyl, acyl or acylamino, or R₃ and R₄ together with the carbon to which they are bound represent C═O, C═S, or C═NR₈, or R₂ together with the nitrogen to which it is bound and R₃ together with the carbon to which it is bound can form a C₄-C₉ heterocycloalkyl, a heteroaryl, a polyheteroaryl, a non-aromatic polyheterocycle, or a mixed aryl and non-aryl polyheterocycle ring;
R₅ is selected from H, C₁-C₆ alkyl, C₄-C₉ cycloalkyl, C₄-C₉ heterocycloalkyl, acyl, aryl, heteroaryl, arylalkyl (e.g. benzyl), heteroarylalkyl (e.g. pyridylmethyl), aromatic polycycles, non-aromatic polycycles, mixed aryl and non-aryl polycycles, polyheteroaryl, non-aromatic polyheterocycles, and mixed aryl and non-aryl polyheterocycles;
n, n₁, n₂ and n₃ are the same or different and independently selected from 0-6, when n, is 1-6, each carbon atom can be optionally and independently substituted with R₃ and/or R₄;
X and Y are the same or different and independently selected from H, halo, C₁-C₄ alkyl, such as CH₃ and CF₃, NO₂, C(O)R₁, OR₉, SR₉, CN, and NR₁₀R₁₁;
R₆ is selected from H, C₁-C₆ alkyl, C₄-C₉ cycloalkyl, C₄-C₉ heterocycloalkyl, cycloalkylalkyl (e.g., cyclopropylmethyl), aryl, heteroaryl, arylalkyl (e.g., benzyl, 2-phenylethenyl), heteroarylalkyl (e.g., pyridylmethyl), OR₁₂, and NR₁₃R₁₄;
R₇ is selected from OR₁₅, SR₁₅, S(O)R₁₆, SO₂R₁₇, NR₁₃R₁₄, and NR₁₂SO₂R₆;
R₈ is selected from H, OR₁₅, NR₁₃R₁₄, C₁-C₆ alkyl, C₄-C₉ cycloalkyl, C₄-C₉ heterocycloalkyl, aryl, heteroaryl, arylalkyl (e.g., benzyl), and heteroarylalkyl (e.g., pyridylmethyl);
R₉ is selected from C₁-C₄ alkyl, for example, CH₃ and CF₃, C(O)-alkyl, for example C(O)CH₃, and C(O)CF₃;
R₁₀ and R₁₁ are the same or different and independently selected from H, C₁-C₄ alkyl, and —C(O)-alkyl;
R₁₂ is selected from H, C₁-C₆ alkyl, C₄-C₉ cycloalkyl, C₄-C₉ heterocycloalkyl, C₄-C₉ heterocycloalkylalkyl, aryl, mixed aryl and non-aryl polycycle, heteroaryl, arylalkyl (e.g., benzyl), and heteroarylalkyl (e.g., pyridylmethyl);
R₁₃ and R₁₄ are the same or different and independently selected from H, C₁-C₆ alkyl, C₄-C₉ cycloalkyl, C₄-C₉ heterocycloalkyl, aryl, heteroaryl, arylalkyl (e.g., benzyl), heteroarylalkyl (e.g., pyridylmethyl), amino acyl, or R₁₃ and R₁₄ together with the nitrogen to which they are bound are C₄-C₉ heterocycloalkyl, heteroaryl, polyheteroaryl, non-aromatic polyheterocycle or mixed aryl and non-aryl polyheterocycle;
R₁₅ is selected from H, C₁-C₆ alkyl, C₄-C₉ cycloalkyl, C₄-C₉ heterocycloalkyl, aryl, heteroaryl, arylalkyl, heteroarylalkyl and (CH₂)ₘZR₁₂;
R₁₆ is selected from C₁-C₆ alkyl, C₄-C₉ cycloalkyl, C₄-C₉ heterocycloalkyl, aryl, heteroaryl, polyheteroaryl, arylalkyl, heteroarylalkyl and (CH₂)ₘZR₁₂;
R₁₇ is selected from C₁-C₆ alkyl, C₄-C₉ cycloalkyl, C₄-C₉ heterocycloalkyl, aryl, aromatic polycycles, heteroaryl, arylalkyl, heteroarylalkyl, polyheteroaryl and NR₁₃R₁₄;
m is an integer selected from 0 to 6; and
Z is selected from O, NR₁₃, S and S(O),
or a pharmaceutically acceptable salt thereof.

3. A combination according to claim 1, wherein (i) the angiotensin receptor blocker (ARB) is valsartan, and (ii) the histone deacetylase (HDAC) inhibitor is N-hydroxy-3-[4-[(2-hydroxyethyl){2-(1H-indol-3-yl)ethyl]-amino]methyl]phenyl]-2E-2-propenamide or N-hydroxy-3-[4-[[[2-(2-methyl-1H-indol-3-yl)-ethyl]-amino]methyl]phenyl]-2E-2-propenamide.

4. A combination according to, claim 2 wherein valsartan is contained in an amount from about 20 to about 640 mg.

5. A combination according to claim 2, wherein valsartan is contained in an amount from about 40 to about 320 mg.

6. A combination according to claim 1, further comprising (iii) a diuretic or a pharmaceutically acceptable salt thereof.

7. A combination according to claim 6, wherein (iii) the diuretic is hydrochlorothiazide.

8. A combination according to claim 7, wherein hydrochlorothiazide is contained in an amount from about 5 mg to about 200 mg.

9. A combination according to claim 7, wherein hydrochlorothiazide is contained in an amount from about 5 mg to about 25 mg.

10. A kit of parts comprising the combination of claim 6 in the form of two or three separate units of the components (i) to (iii).

11. A method of treatment and/or prevention of cardiovascular disorders comprising administering a therapeutically effective amount of the combination according to claim 1 to a mammal in need of such treatment.

12. A method according to claim 11 wherein the cardiovascular disorder is selected from the group consisting of hypertension, heart failure such as (acute and chronic) congestive heart failure, pathological cardiac hypertrophy, left ventricular dysfunction and hypertrophic cardiomyopathy, diabetic cardiac myopathy, supraventricular and ventricular arrhythmias, atrial fibrillation, atrial flutter, detrimental vascular remodeling, myocardial infarction and its sequelae, atherosclerosis, angina (whether unstable or stable), renal insufficiency (diabetic and non-diabetic), heart failure, angina pectoris, diabetes, secondary aldosteronism, primary and secondary pulmonary hypertension, renal failure conditions, such as diabetic nephropathy, glomerulonephritis, scleroderma, glomerular sclerosis, proteinuria of primary renal disease, and also renal vascular hypertension, diabetic retinopathy, the management of other vascular disorders, such as migraine, peripheral vascular disease, Raynaud's disease, luminal hyperplasia, cognitive dysfunction (such as Alzheimer's), glaucoma, stroke, right ventricular hypertrophy, e.g. as associated with pulmonary hypertension, cardiac fibrosis, blood pressure-related cerebrovascular disease, end-organ damage, including that to the kidneys, vasculature and neural systems, for example nephropathy, vasculopathy and neuropathy and diseases of the coronary vessels.

13. A method according to claim 11 wherein the cardiovascular disorder is selected from the group consisting of heart failure such as (acute and chronic) congestive heart failure and pathological cardiac hypertrophy.

14. A commercial package comprising
(i) a pharmaceutical composition of an angiotensin receptor blocker (ARB),
(ii) a pharmaceutical composition of histone deacetylase (HDAC) inhibitor, and
(iii) optionally a pharmaceutical composition of a diuretic, in the form of two or three separate units of the components (i) to (iii), together with instructions for simultaneous, separate or sequential use thereof for the treatment or prevention of a condition or disease selected from the group consisting of hypertension, heart failure such as (acute and chronic) congestive heart failure, pathological cardiac hypertrophy, left ventricular dysfunction and hypertrophic cardiomyopathy, diabetic cardiac myopathy, supraventricular and ventricular arrhythmias, atrial fibrillation, atrial flutter, detrimental vascular remodeling, myocardial infarction and its sequelae, atherosclerosis, angina (whether unstable or stable), renal insufficiency (diabetic and non-diabetic), heart failure, angina pectoris, diabetes, secondary aldosteronism, primary and secondary pulmonary hypertension, renal failure conditions, such as diabetic nephropathy, glomerulonephritis, scleroderma, glomerular sclerosis, proteinuria of primary renal disease, and also renal vascular hypertension, diabetic retinopathy, the management of other vascular disorders, such as migraine, peripheral vascular disease, Raynaud's disease, luminal hyperplasia, cognitive dysfunction (such as Alzheimer's), glaucoma, stroke, right ventricular hypertrophy, e.g. as associated with pulmonary hypertension, cardiac fibrosis, blood pressure-related cerebrovascular disease, end-organ damage, including that to the kidneys, vasculature and neural systems, for example nephropathy, vasculopathy and neuropathy and diseases of the coronary vessels. hyperplasia, cognitive dysfunction (such as Alzheimer's), glaucoma and stroke

15. A commercial package according to claim 14, wherein (i) the angiotensin receptor blocker (ARB) is valsartan; (ii) histone deacetylase (HDAC) inhibitor is N N-hydroxy-3-[4-[(2-hydroxyethyl){2-(1H-indol-3-yl)ethyl]-amino]methyl]phenyl]-2E-2-propenamide or N-hydroxy-3-[4-[[[2-(2-methyl-1H-indol-3-yl)-ethyl]-amino]methyl]phenyl]-2E-2-propenamide; and (iii) the optional diuretic is hydrochlorothiazide.

16. A commercial package according to claim 14, wherein the angiotensin receptor blocker (ARB) (i) and the diuretic (iii) are present in the form of Co-DIOVAN® or wherein the angiotensin receptor blocker (ARB) (i) is present in the form of DIOVAN®.

17-18. (canceled)

19. A method according to claim 11 wherein the cardiovascular disorder is selected from the group consisting of heart failure such as (acute and chronic) congestive heart failure and pathological cardiac hypertrophy.
